(19) United States
(12) Patent Application Publication
(10) Pub. No.: US 2004/0268427 A1
(43) Pub. Date: Dec. 30, 2004

(54) Polymerase-mediated regulation of polynucleic acids

(76) Inventor: Paul Diamond, Secaucus, NJ (US)

Correspondence Address: Paul Diamond, 942 Schopmann Drive, No. 2, Secaucus, NJ, 07094, US

(21) Appl. No.: 10/354,903

(22) Filed: Jan. 29, 2003

Related U.S. Application Data
(60) Provisional application No. 60/352,705, filed on Jan. 29, 2002.

(51) Int. Cl. A61K 48/00; C12N 15/85

(52) U.S. Cl. 800/21; 435/455

(57) ABSTRACT

The invention provides methods and systems for controlling the expression and, in general, the cellular activity of preselected polynucleic acid molecules. The invention also provides methods and systems for genetically modifying cells and multi-cellular organisms to impart resistance to viruses. The invention further provides methods and systems for genetically modifying cells and multi-cellular organisms so that they diagnostically report viral infection. The invention involves rendering target polynucleic nucleic acid molecules to be functional templates for at least one template-directed polynucleic acid polymerase so that utilization of the polynucleic acid molecule as a template by the polymerase modulates the activity of the targeted polynucleic acid molecule.

This application claims priority to U.S. provisional application Ser. No. 60/352,705 filed Jan. 29, 2002, which is hereby incorporated by reference in its entirety.

### FIELD OF THE INVENTION

The invention relates to methods and systems for controlling the activity of preselected polynucleic acid molecules.

### BACKGROUND OF THE INVENTION

U.S. Pat. No. 5,597,697, hereby incorporated by reference in its entirety, discloses polymerase-based methods for controlling the activity of preselected polynucleic molecules by rendering these molecules templates for the template-directed polymerization of nucleic acids.

RNA silencing is a cellular, sequence-specific RNA degradation mechanism that occurs in a broad range of eukaryotic organisms including fungi (quelling), animals (RNA-interference, RNAi) and plants (post-transcriptional gene silencing, PTGS). In these organisms, RNA silencing is triggered by double-stranded RNA (dsRNA) and requires a conserved set of gene products. Recent reviews of RNA silencing in plants include Matzke et al, Science 293, 1080-1083 (2001); Vance and Vaucheret, Science 292, 2277-2280 (2001); Voinnet, Trends Genet., 17, 449-459 (2001) and Waterhouse et al., Nature, 411, 834-842 (2001), and in fungi or animals, Cogoni and Macino, Current Opin. Genet. Dev. 10, 638-643 (2000;) Bernstein et al. Nature, 409, 363-366 (2001); Carthew, Curr. Opin. Cell Biol. 13, 244-248 (2001) and Zamore, Natl. Sruct. Biol. 8, 746-750 (2001), which are hereby incorporated by reference.

The mechanism of RNA silencing is proposed to involve processing of a “long” inducing dsRNA molecule into dsRNA fragments of 21 to 25 nucleotides. The enzyme Dicer, a member of the RNAse III family of dsRNA ribonucleases, processively cleaves an inducer dsRNA. Successive cleavage events degrade the 21-25 nucleotide fragments into 19-21 bp duplexes (small interfering RNAs, “siRNAs”) having 2-nucleotide 3′ overhangs. The siRNAs are proposed to then associate with the RNA-induced silencing complex (RISC) to target and degrade mRNA molecules having complementarity to the siRNA, wherein the siRNA strands serve as primers to render a target mRNA a template for template directed polymerization of ribonucleotides and further RNAse III-type dsRNA nuclease activity to inactivate the protein coding activity of the target molecule and generate further siRNAs.

Synthetic siRNAs are capable of inducing an RNA-silencing response in human and other mammalian cells that are not substantially capable of processing dsRNA to siRNA. Short RNA hairpins (stem-loop structures) can also be used to induce RNA silencing against RNA targets having regions of complementarity to at least one strand of the stem sequence of such the hairpin. (Discussed in Piccin et al, Nucleic Acids Res. Vol. 29, No. 12. e55, 2001, and Wesley et al., Plant J 27(6): 581-590, 2001, each incorporated by reference herein.) RNA:DNA hybrid molecules are also reported to be capable of triggering the RNA-silencing mechanism, as disclosed in published U.S. patent application Ser. No. 09/920,342.

### SUMMARY OF THE INVENTION

An essential feature of the invention is the incorporation of a functional polymerase binding site sequence (PBS) into a nucleic acid molecule that confers a discernible characteristic (for example via its sequence specific activity) such that the incorporation of the PBS renders the nucleic acid molecule a functional template for a given RNA or DNA template-directed nucleic acid polymerase. In the presence of the polymerase and primer, catalytic extension of the strand of nucleic acids complementary to the template occurs, resulting in the modulation (decrease or increase) of the characteristic-conferring activity of the reporter-template molecule.

The invention provides methods and compositions for inhibiting the expression of, or other activities of, selected polynucleic acid molecules, for example, specific cellular and/or viral mRNA transcripts. The invention further provides methods and compositions for inhibiting the replicative ability of specific polynucleic acid molecules within a cell, for example viral genomes such as, but not limited to, plus or minus strand viral genomic RNA molecules.

The invention also provides methods and compositions that render a cell or multi-cellular organism resistant to viral infection. The invention further provides diagnostic methods and compositions for detecting the presence of a preselected virus in a sample.

The invention further provides methods and compositions for regulating the expression of a preselected gene in a cell, e.g. a transgene, by selectively rendering a transcriptional regulatory protein controlling the expression of the preselected gene to be a functional template for a template directed polynucleic acid polyemerase.

### DETAILED DESCRIPTION OF THE INVENTION

The term “polymerase binding site” (abbreviated PBS) as used herein is defined as a sequence element in a nucleic acid molecule that renders the molecule a template for a specified nucleic acid polymerase by mediating interaction between the polymerase and the nucleic acid molecule. Accordingly, primer binding sequences, promoter sequences, and origins of replication, as known to those in the field, are examples of polymerase binding sites. As illustrated in Orr et al, The Journal of Biological Chemistry, 267, 4177-4182 (1992), with a number of polymerases, including HIV-RT, primer extension assays can be performed utilizing heterologous PBS sequences (those not normally used by a given polymerase) as long as there is sufficient complementarity between a region of the template molecule and the primer molecule used. An example is the “combined” template-primer molecule poly(rA)-oligo(dT).sub.12-18. Such a template sequence, which is complementary to the primer in question, also constitutes a polymerase binding site as defined herein.

An essential feature of the invention is the incorporation of a functional polymerase binding site sequence (PBS) into a nucleic acid molecule which is chosen for its ability to confer a discernible characteristic (for example via its sequence specific activity) such that the incorporation of the PBS renders the nucleic acid molecule a functional template for utilization by a predetermined RNA or DNA template-directed nucleic acid polymerase. In the presence of the polymerase, suitable primer molecules, and any necessary accessory molecules, catalytic extension of the strand of nucleic acids complementary to the template occurs, resulting in a partial or total elimination of (or increase in) the characteristic conferring activity of the reporter-template molecule described due to the effects of the complementary strand or other polymerase-mediated effects.

A reporter template polynucleic acid molecule according to the invention is a polynucleic molecule (DNA or RNA) that confers some discernable characteristic, in vitro and/or in vivo, for example a cell phenotype altering activity.

The direct incorporation of a PBS sequence or sequences into, for example, an RNA reporter strand may be achieved by the ligation of double stranded DNA restriction enzyme fragments containing the PBS sequence into appropriate restriction sites of a double stranded DNA molecule which is a template for the transcription of an RNA reporter strand by a DNA-dependent RNA polymerase. Alternatively the incorporation of the PBS sequence can be achieved through site-directed mutagenesis of such a DNA molecule, the total chemical synthesis of the novel RNA reporter molecule (by, for example, the phosphoramidite method using an Applied Biosystems Model 392 DNA/RNA Synthesizer), or by any other method known in the field.

Examples of the activity of the reporter-template RNA molecule include, but are not limited to, the following types. The RNA can be a messenger RNA (mRNA) coding for a protein that confers a discernible characteristic. In this case the preferable form of the assay is cell-based such that the reporter-template RNA is produced within a suitable host cell along with the components necessary for the extension of the nucleic acid strand complementary to the reporter-template molecule, specifically the appropriate RNA-dependent nucleic acid polymerase and its necessary primer molecule if any. The characteristic-conferring protein may be, but is not limited to, an enzyme catalyzing a color reaction such as beta-galactosidase (catalyzes the chromogenic conversion of the substrate 5-bromo-4-chloro-3-indolyl-beta-D-galactoside), an enzyme conferring antibiotic resistance, such as beta-lactamase which confers resistance to ampicillin, or an enzyme conferring the metabolic complementation of an auxotrophic state. For cell-based and multi-cellular organism-based embodiments of the invention the characteristic conferring activity can relate to any alteration of cellular phenotype.

Alternative positions for the incorporation of a PBS into an mRNA molecule with respect to the positions of the start codon and stop codon of the mRNA molecule include the following. The PBS can be incorporated into the 3′ non-coding sequence of the mRNA. The PBS can also be incorporated into the protein coding sequence of the mRNA. Incorporation of the PBS into the coding region is appropriate as long it does not change the coding sequence such that a non-functional characteristic-conferring protein is produced. The PBS may also be incorporated within one or more introns within an mRNA transcript. Finally, the PBS can be incorporated into the 5′ non-coding region of the mRNA. This region contains the ribosome binding site and other regulatory sequences. Polymerase activity initiated by binding at the PBS in any of these regions can prevent translation of the mRNA by at least one of several mechanisms such as a direct block of sense strand translation by the complementary strand, the complementary strand-facilitated degradation and/or modification of the sense strand by enzymes such as RNAse H (specifically hydrolyzes the phosphodiester bonds of RNA in RNA:DNA duplexes to generate products with 3′ hydroxyl and 5′ phosphate ends.) and RNAse III (degrades RNA:RNA duplexes), e.g., E. coli RNAse III, interference with the RBS by formation of its complementary strand and/or its degradation. Binding of the polymerase to its PBS without the ensuing synthesis of the complementary strand may also prevent translation due to steric interference with the translational apparatus, in particular this may be expected to occur if the PBS sequence is located near the RBS or between the RBS and the end of the protein coding sequence, but not in the 3′ non-coding region or necessarily at other sites 5′ to the RBS in the 5′ non-coding region. The choice of PBS incorporation sites will be partly influenced by the need or lack of need to specifically determine the differential effects agents have on polymerase/PBS binding and on complementary strand extension by the polymerase. The invention is not limited by the use of single or multiple PBS sequences incorporated into one or more of the alternate positions described in the same mRNA molecule.

The invention is not limited by the means of incorporation of a PBS sequence into a polynucleic acid molecule to render it a template. In addition to altering the sequence of polynucleic acid molecule (or gene encoding it), these means also include the generation of a heterologous PBS by providing a primer sequence sufficiently complementary to a region of the polynucleic acid molecule without any modification of the template itself. The invention is not limited by the singularity, multiplicity, or position of PBS sequences incorporated into a reporter-template or other target polynucleic acid molecule nor is the invention limited by the nature of the discernible characteristic conferred by the reporter-template molecule or any target template molecule according to the invention.

For example, the reporter-template RNA molecule is a regulatory RNA controlling the expression and/or other activity of one or more genes or gene products that confer a discernible characteristic. In this case also, the components comprising the invention are preferably produced within a suitable host cell.

In another example, the reporter-template RNA molecule is a catalytic RNA molecule that confers a discernible characteristic directly, by virtue of its catalytic activity. This activity may include, but is not limited to the catalysis of color reactions. The catalytic reporter-template RNA molecule described can also be an RNA:DNA hybrid in which the incorporated primer and perhaps some other sequence of the molecule is composed wholly or partly of deoxyribonucleic acids.

The preceding discussion and examples also illustrate the manner in which any specific RNA molecule, whether naturally-occurring or genetically engineered, including but not limited to specific niRNA molecules and viral genomic RNA molecules, can be targeted according to the invention by one or more RNA-dependent nucleic acid polymerases, i.e., RNA-dependent RNA polymerases or reverse transcriptases, to inhibit one or more activities of the targeted RNA molecule.

In reporter template embodiments related to DNA-dependent DNA Polymerases, the reporter-template molecule can, for example, be a catalytic DNA molecule that confers a discernible characteristic directly, by virtue of its catalytic activity. This embodiment is analogous to that for RNA-dependent nucleic acid polymerases in which the reporter-template is a catalytic RNA. Functional interaction of a DNA-dependent DNA polymerase and the reporter-template reduces the catalytic activity of the reporter-template and provides an assay for inhibitors and activators of polymerase activity as previously described.

In reporter template embodiments related to DNA-dependent RNA Polymerases the reporter-template may be a single stranded, partially double stranded, or double stranded DNA molecule that confers a discernible characteristic, for example, in a direct manner via its sequence specific catalytic activity. Such a reporter-template contains the promoter sequence and other sequences necessary to direct transcription of RNA. Functional interaction of a DNA-dependent RNA polymerase and the reporter-template reduces the latter's catalytic activity.

The cell-based embodiments of the invention may comprise any suitable host cell as long as the components of the assay are functional in the cell type in question. These host cell types may include but are not limited to mammalian cells, avian cells, fish cells, insect cells, plant cells, yeasts and bacteria.

RNA-degrading and/or modifying enzymes that may be used in accordance with the invention, as described, may for example be endogenous to the host cells or introduced by genetic methods to the host cells. In the case of in vitro, non-cell-based embodiments of the invention, enzymes such as nucleases can be directly provided to the composition or mixture comprising the invention.

### EMBODIMENTS RELATED TO SYSTEMS AND METHODS FOR CONTROLLING THE ACTIVITY OF POLYNUCLEIC ACID MOLECULES WITHIN CELLS AND MULTI-CELLULAR ORGANISMS

In this embodiment, the invention is used to control gene expression and, in general, the activity of any nucleic acid strand of interest. By operably linking the activity of a nucleic acid polymerase to a nucleic acid strand of interest the expression of, or other activity of, the nucleic acid strand can be controlled by the addition and subtraction of the polymerase itself, cofactors of the polymerase, or inhibitors and activators of the polymerase in question. In one embodiment of the invention, the cellular expression of a specified protein is controlled by operably linking the activity of an RNA-dependent nucleic acid polymerase to an mRNA molecule coding for the protein.

In another embodiment of the invention, the polynucleic acid molecule rendered subject to polymerase-mediated regulation is a viral replicative polynucleic acid molecule and the activity inhibited by the polymerase is the replicative activity of the polynucleic acid.

In still another embodiment of the invention, the polynucleic acid molecule rendered subject to polymerase-mediated regulation is a viral mRNA molecule or viral regulatory RNA molecule, and the activity inhibited by the polymerase is the viral function of the protein encoded by the viral mRNA molecule or the viral function of the viral regulatory molecule.

In a further embodiment of the invention, the polynucleic acid molecule rendered subject to polymerase-mediated regulation comprises a polynucleic acid aptamer (DNA or RNA) molecule having specific binding activity to one or more ligand molecules, as known in the art. Thus, the invention provides a method for reducing the binding between a polynucleic acid aptamer and a ligand to which the aptamer has characteristic binding affinity by rendering the aptamer a template for the template directed polymerization of nucleic acids by a template directed nucleic acid polymerase. Said polymerization can reduce the binding between the aptamer and its ligand by disrupting the characteristic ligand-binding secondary structure of the aptamer and/or by resulting in the degradation of the aptamer.

As disclosed above, in other embodiments of the invention the polynucleic acid molecule rendered subject to polymerase-mediated regulation is a regulatory polynucleic acid molecule or a catalytic polynucleic acid molecule.

According to the invention, the polymerase or polymerases selected to inhibit the expression or other activity or function of a targeted polynucleic acid can be provided to the cell(s) in several ways. First, the polymerase may be a viral polymerase that is provided to the cell as a result of infection of the cell by a virus via, for example, (i) translation of polymerase-encoding mRNA which is part of an infecting virion, (ii) translation of polymerase-encoding mRNA which is transcribed from the viral genome within the cell and/or (iii) when the viral polymerase is a component of the virion itself (e.g., as in the case of HIV) by direct delivery to the cell as a result of infection of the cell by the virus. Second, the cell may be genetically modified to express a suitable polynucleic acid polymerase, which polymerase is not otherwise expressed by the cell at all or at least not ordinarily expressed to a sufficient level to effectuate the desired polymerase-mediated inhibition. Third, the polymerase can be a polymerase endogenous to the cell(s) but which under normal cellular conditions is not substantially directed to a preselected polynucleic acid molecule for which inhibition is desired.

### INHIBITION OF MULTIPLE TARGET MOLECULES

Plant and animal cells can be engineered to express several different sequence specific primers targeting different transcripts and/or genomic elements for one or more specific viruses thereby imparting multiple viral resistances to the cell(s).

### ENDOGENOUS OR EXOGENOUS POLYMERASES

A e polymerase selected for use according to the invention to inhibit the activity of a target polynucleic acid molecule can, for example, be endogenous to the cell or multi-cellular organism, can be provided by a virus infecting the cell(s), or can be provided to the cell(s) or multi-cellular organism by genetically modifying the cell(s) or multi-cellular organism, according to standard methods, to express the polymerase. In some cases an identified cell type may be known to possess a desired, endogenous, template-directed nucleic acid polymerase activity although the enzyme responsible for the activity has not been identified. In this case, selecting a template directed nucleic acid polymerase for use according to the invention can comprise constituting the elements of the invention within cells of the cell type.

### SELECTING RNA OR DNA POLYMERASES

For the suppression of mRNA transcripts or other RNA molecules, both RNA-directed DNA polymerases and RNA-directed RNA polymerases are suitable according to the invention. For the suppression of viral genomic replication in particular, the target suppressing polymerase selected may, for example, be of the type not utilized in normal viral replication. For example, the replication intermediates of RNA viruses which normally utilize a viral RNA-directed RNA polymerase for replication can be targeted by an RNA-directed DNA polymerase so that a replication-incompetent RNA:DNA hybrid is formed.

For example, in one embodiment of the invention, hoofed mammals susceptible to Foot and Mouth Virus (FMV) are genetically modified to make them resistance to infection by FMV. FMV is a single stranded, plus strand RNA virus. Normal replication of the FMV genome is dependent on an RNA-dependent RNA polymerase. There is no DNA replication intermediate in the FMV replication cycle. Thus, in one implementation of the invention, FMV susceptible animals are genetically modified to express a Reverse Transcriptase (RT) and primer molecule with a region of complementarity to the FMV plus strand which, together, target and inhibit replication of the plus strand RNA of FMV. In contrast to the double stranded RNA intermediate characteristic of FMV replication, FMV is not at all naturally equipped to replicate or serve as a template for transcription when the plus strand is complexed with complementary DNA to form an RNA:DNA hybrid, such as that formed by interaction with the RT.

RNAses or DNAses, integral to the polymerases, endogenous to the cell or engineered to be expressed in the cell, may also be employed according to the invention to degrade the target molecule as the selected polymerase utilizes the target as a template or at any point thereafter. Hence, in the FMV example above, if an RT having an integral RNAse H, such as HIV-RT, is used, the plus strand FMV RNA will be digested during its reverse transcription. Examples of reverse transcriptase enzymes that lack RNAse activity and that can be used according to the invention include the various RNAse H domain/activity deficient mutants of HIV-RT that are known in the art. Members of the Dicer endonuclease family (Bermstein et al. Nature, 409, 363-366, 2001; Hutvagner et al. Science, 293, 834-838, 2001; Nicholson et al. Mamm Genome, 13(2), 67-73, 2002) of RNAse III type endonucleases are examples of nucleases suitable for digesting double stranded RNA molecules formed as a result of rendering an RNA molecule a template for an RNA-dependent RNA polymerase according to the invention.

Any suitable type of primer molecule can be used to specifically prime the desired polymerase activity. One method of targeting a reverse transcriptase such as HIV-RT or HIV-RT derived polymerase to a target preselected polynucleic acid molecule in a cell is to provide the cell with the expression of the HIV-RT primer, tRNA^{Lys-3} modified so that its 3′ polynucleotide sequence is complementary to a sequence of the target molecule. Such expression may be provided by introduction of a primer transgene into a cell, or multi-cellular organism, as part of an expression cassette, and/or by genetically modifying a preexisting cellular gene for tRNA^{ys-3}, for example, by homologous recombination techniques. The polymerase binding site and primer requirements for HIV-RT are disclosed in Weiss et al, Gene, 111, 183-197 (1992) and Kohlstaedt and Steitz, Proc. Natl. Acad. Sci. USA 89, 9652-9656 (1992). Those for HBV-RT are disclosed in Wang and Seeger, Cell, 71, 663-670 (1992).

Many different RNA-dependent RNA polymerases (RdRPs) can be used according to the invention. These include, for example, RdRPs of RNA viruses and cellular RdRPs of plants. Those skilled in the art will appreciate that any sort of cell can be genetically engineered to express a selected RdRP gene. Any suitable type of primer molecule can be used to specifically prime the desired RdRP activity according to which RdRP is selected. Many RNA virus genomes and satellite RNA virus genomes have a 3′ integral primer, which comprises secondary structure resembling tRNA. Examples of viruses with tRNA-like structure at the 3′ end of their genome include: Tymoviruses (e.g. turnip yellow mosaic virus; Bromoviruses (e.g. brome mosaic virus); Cucumoviruses (e.g. cucumber mosaic virus), Hordeiviruses (e.g. barley stripe mosaic virus), Tobamoviruses (e.g. tobacco mosaic virus), Tobraviruses (e.g. tobacco rattle viruses). One method of targeting an RDRP such as BMV-RdRP, to a target preselected RNA molecule in a cell is to provide the cell with the expression of a primer molecule based on the 3′ integral primer of the virus and modified so that its 3′ polynucleotide sequence is complementary to a sequence of the target molecule to be rendered a template.

### MISPRIMING TECHNIQUES

Cells engineered with primers that redirect a viral polymerase to an “inappropriate starting point” along the viral genome, i.e., different than the primer binding site characteristically used by the virus, may be used to prevent the formation of full length viral replication intermediates. For example, engineered primers binding downstream (in the 3′ direction with respect to the primer) of the virus' normal primer binding site cause a non-full length primer extension product to be formed rather than the virally-normal, replication-competent product. As a result, polymerase mediated synthesis of the full-length “virally-normal” product is at least partially inhibited. In one embodiment of this method, the viral polymerase contains an integral or closely associated nuclease, such as an RNAse H, that degrades the template during template-directed polymerization. In this manner, synthesis of the virally-normal, full-length product is precluded since some part of the template strand downstream of the PBS normally used by the virus is degraded, thereby preventing the formation of the characteristic full-length product from the characteristic PBS. An example of a polymerase with an RNAse H domain and which uses a characteristic viral PBS is HIV Reverse Transcriptase (HIV-RT).

### CONTROL OF ACTIVITY OF PRESELECTED CELL-ENCODED POLYNUCLEIC ACID MOLECULES BY REDIRECTION OF VIRAL POLYMERASE ACTIVITY TO THE CELL-ENCODED MOLECULES

Cells may be engineered to express specific primer molecules that may the serve as templates, so that, in infected cells, the activity of the preselected polynucleic acid molecules coded by the genes (specific mRNAs molecules, regulatory RNA molecules, etc.) is modulated (reduced or increased) as a result of the functional interaction between the viral polymerase and the polynucleic acid molecule.

In another embodiment of the invention, a cellular gene (of endogenous origin or foreign origin with respect to the cell) is genetically modified so that the single stranded polynucleic acid molecule product of the gene (ssRNA or ssDNA) comprises a functional polymerase binding site sequence (for example, as a result of altering the sequence of the gene or by providing for the cellular expression of a suitable primer) which renders the polynucleic acid molecule a template for the viral polymerase in the presence of the viral polymerase in the cell, thereby reducing the activity of the polynucleic acid molecule.

### CELLULAR RESISTANCE TO VIRUS VIA REDIRECTION OF VIRAL POLYMERASE ACTIVITY TO VIRAL POLYNUCLEIC ACIDS OR POLYNUCLEIC ACIDS PRODUCED USING THE VIRAL GENOME AS A TEMPLATE

Cells may be engineered to express specific primer molecules so that, in infected cells, preselected viral RNA molecules are targeted and their normal activity inhibited by at least one viral polymerase provided by the virus itself. In this context “provided” by the virus means that the polymerase polypeptide is already present in the virion when it enters the cell or that the virion contains mRNA coding for the polymerase, which mRNA is translated once in the cell or that the viral genome is capable of serving as a template for the transcription of mRNA coding for the polymerase, which mRNA is normally transcribed and translated in the cell. In this embodiment of the invention, the viral polymerase is directed to viral polynucleic acids or regions thereof, which do not normally serve as templates for the viral polymerase. In this manner, the normal activity of the viral nucleic acids is inhibited. For example, in the case of a virus providing a reverse transcriptase or an RNA-dependent RNA polymerase, a cell or multi-cellular organism can be genetically modified to express primer molecules that direct the polymerase to use an mRNA transcript of the virus as a template for the template directed polymerization of nucleic acids. In this manner, translation of the viral mRNA transcript is inhibited.

### POLYMERASE-MEDIATED VIRAL INFECTION DETECTION SYSTEMS

A biological assay for the presence or absence of particular viruses in a sample or in the environment in general is provided according to the invention by modifying cells to express cellular transcripts which have reporter activity and which are operably-linked to the viral polymerase activity when the polymerase is provided by infection of the cell, so that the reporter activity is inhibited by the viral polymerases of the particular infecting virus(es). This viral infection detection system may, for example, be employed in a laboratory assay format utilizing test cells, i.e., a test cell line subjected to samples. These cells may be of the type or derived from the type of cells that are naturally infected by the particular viruses for which the assay is developed. Alternatively, cells which are not normally susceptible to infection by the particular virus(es) for which it is desired to develop assay cells, may be engineered to have the appropriate susceptibility to infection. This can be achieved by various methods known in the art such as, but not limited to, engineering the subject cells to express a virus receptor protein or receptor glycoside which is otherwise missing from the subject cell or expressed on at a low level by the cell. Those skilled in the art will understand that the method will be tailored to the specific virus and cell type in question.

Multi-cellular organisms can also be genetically modified to embody this type of viral detection system generally, or in one or more selected tissues or cell types. In one embodiment of the invention, a plant is engineered to embody the viral detection system. A suitable reporter gene for a plant can be, for example, a gene conferring a particular pigmentation or coloration, the absence of the expression of the gene being readily discemable. In this manner, substantially real-time monitoring of pathogen activity in a field of crops can be accomplished and appropriate steps to limit further crop damage can then taken. In another embodiment of the invention, animals such as, but not limited to, livestock can be genetically modified to similarly embody the viral detection system in one or more tissues and hence report the presence of viral infection.

In one method of the invention, agricultural crops in the same field or commonly raised animals generally embody a viral infection detection system according to this or any viral infection detection embodiment of the invention. In this manner, real- time monitoring of pathogen activity can be accomplished and appropriate steps to limit further damage, such as destruction of the affected section of crops or segregation of affected livestock, can then taken. In another method according to the invention “detection” organisms embodying a viral infection detection system according to the invention are provided within a field of agricultural crops or among a group of commonly raised livestock that generally do not embody such a detection system.

One embodiment of the invention provides a diagnostic method, and cell compositions therefor, for determining the presence or absence of a pre-specified virus in a sample comprising the steps of: providing tests cells genetically engineered so that a preselected reporter template molecule expressed within the cells becomes a functional template of a template directed nucleic acid polymerase as a result of infection of the cells by virus present in the samples; contacting a sample potentially containing virus with the test cells; and determining whether the discemable characteristic-conferring activity of the reporter-templates of the test cells is modulated, thereby indicating presence of the virus in the sample.

A related diagnostic embodiment comprises performing the steps in parallel with the test sample and a negative control sample not containing the virus, and comparing the result obtained using the test sample to the results obtained from the negative control sample to determine whether the test sample contains the virus. Still another embodiment of the diagnostic method comprises performing the steps in parallel with the test sample and a positive control sample containing a control virus to determine whether the test sample contains the virus. For safety, when the virus for which testing is being performed is a pathogenic virus of humans, other animals or plants, the virus used as the positive control need not actually be pathogenic but merely needs to resemble the actual pathogenic virus with respect to mimicking its performance in the assay. In a still further related -embodiment, the diagnostic method is performed using the test sample and both the positive and negative control samples.

The discemable characteristic-conferring activity of the reporter template may be of any detectable sort including, but not limited to, directly or indirectly repressing the expression of another gene that has reporter activity by, for example, virtue of coding for a protein having reporter or selectable marker activity as known in the art, whereby said repression is relieved as a result of the reporter template being made to serve as a template upon viral infection.

The test cells of these diagnostic embodiments may be cultured in any appropriate format, including, but not limited to, liquid suspension culture or culture on a surface submerged in growth media or on a surface of growth media. Further, the steps of the diagnostic method may, for example, be performed in one or more of any sort of appropriate tube, well plate, vessel or container as known in the art.

It should be understood that in the performance of the steps of the diagnostic method, changes in the reporter activity of the tests cells caused by virus can include the change caused by the original virus particles in the sample and also progeny virus which propagates from this original virus. Thus, according to the invention, after treating the tests cells with a sample, the mixture can optionally be incubated for varying amounts of time to allow for viral propagation to occur. In another embodiment, virus that may exist in a sample can be propagated in other cells before testing to amplify the amount of virus in the original sample. The cells used for propagation of the virus in this embodiment need not be test cells, but can be any sort of cells permissive for propagation of the virus. In a related embodiment, the propagator cells, having been incubated with the original sample, are separated from the putatively-propagated virus containing mix/culture and the cell-free mix is then introduced to the test cells.

The cell-based diagnostic assay embodiments of the invention can benefit in sensitivity by the use of test cells which are highly prone to infection by the subject virus(es) for which the diagnostic assay is designed. Accordingly one embodiment of the invention provides that the test cells are mutated and selected to be more highly prone to infection by the subject virus(es). Such mutation may, for example, be random in response to a mutagenic treatment followed by selection or may involve genetic engineering. For example, a cell being designed for use as a test cell can be genetically engineered to express a higher level of the receptor that the subject virus(es) uses to enter the cell.

### TRANSCRIPTIONAL REGULATORY PROTEIN EMBODIMENTS OF THE INVENTION

In this embodiment of the invention, the RNA molecule made to be a template for a viral polymerase is an mRNA coding for a transcriptional activator protein or a transcriptional repressor protein that activates or represses, respectively, the expression of one or more other preselected genes of any sort including, but not limited to: reporter genes, lethal/toxic genes, essential genes, cell phenotype altering genes, herbicide resistance genes, and genes which themselves code for a transcriptional regulatory protein controlling the expression of still one or more other genes. Further, the invention provides that the preselected genes are of the sort found in nature to be under the control of the regulatory protein or genes not found in nature under control of the regulatory protein.

### DEREPRESSION OF EXPRESSION EMBODIMENTS OF THE INVENTION

In one embodiment of the invention, an RNA molecule made to be a template for a viral polymerase is an mRNA coding for a transcriptional repressor protein which represses the expression, or is engineered to repress the expression of, one or more other preselected genes such as, but not limited to, a reporter gene or a suicide gene which causes an infected cell to die, thus limiting spread of the viral infection within a multi-cellular organism or to other organisms.

For example, a cell can be genetically modified so that the expression of the luciferase gene is repressed by a repressor protein that is expressed within the cell. The repressor protein used may be endogenous to the cell or expressed within the cell as a result of genetically modifying the cell. In any case, in this embodiment, the repressor protein transcript is operably-linked to the activity of one or more selected viral polymerases in the fashion of the invention. Upon infection of the cell(s), direction of the viral polymerase to this repressor gene transcript prevents its expression, thereby leading to derepression of, in this example, the luciferase gene. Addition of the luciferase substrate and action thereon by the luciferase enzyme, if its expression has been de-repressed, will cause light to be emitted thus indicating the derepression. Another example of a reporter gene that can be used is green fluorescent protein (GFP) or any of its numerous derivatives known in the art. In this manner, a test system for the presence or absence of particular viruses can be provided.

The repressor protein coded by the repressor mRNA transcript may be an inhibitory transcription factor capable of directly interacting with the regulatory sequences of the repressed gene, whether endogenous or engineered, as known in the art or may indirectly interact with other biomolecules present in the cell to repress the repressed gene. For example, for plant embodiments of the invention, the Tn10 tet repressor system, as described in Gatz and Quail (1988); Gatz, et al. (1992), can be adapted for use according to the invention. In this system, a modified Cauliflower Mosaic Virus (CaMV) 35S promoter containing one or more, e.g. three, tet operons is used; the Tn10 tet repressor gene produces a repressor protein that binds to the tet operon(s) and prevents the expression of the gene to which the promoter is linked. The presence of tetracycline inhibits binding of the Tn10 tet repressor to the tet operon(s), allowing free expression of the linked gene. Gatz and Quail, “Tn10-encoded tet repressor can regulate an operator-containing plant promoter,” Proc. Natl. Acad. Sci. USA, 85:1394-1397 (1988) and Gatz, et al., “Stringent repression and homogenous derepression by tetracycline of a modified CaMV 35S promoter in intact transgenic tobacco plants,” The Plant Journal, 2:397-404 (1992), hereby incorporated by reference. However, the present invention is not concerned with regulation of the system by tetracycline, although such regulation for which the prior system was designed may optionally be left intact according to the present invention. Instead, according to the invention, the transcript for the repressor protein is made to serve as a template for the viral polymerase, thereby reducing the production of the repressor protein and activating expression driven by the modified CaMV 35S promoter.

Tetracycline responsive promoter systems known in the art for mammalian cells can similarly be adapted for use according to the invention. For example, U.S. Pat. Nos. 5,723,765 and 6,242,667 disclose suitable repressor systems, and are hereby incorporated by reference in its entirety. Those skilled in the art will appreciate that there are many characterized natural repressor and genetically engineered transcriptional activator protein and transcriptional repressor protein systems for plant and animal cells that can be routinely adapted to function according to the invention as described herein. Those skilled in the art will further understand that any gene of interest may be placed under the control of any operable transcriptional repressor or activator by genetically modifying a cell (stably or transiently) and that the repression or activation can be selectively modulated, according to the invention, by rendering the mRNA transcripts coding for the transcriptional regulator protein as functional templates for a template directed polynucleic acid polymerase.

One related embodiment provides introducing a series of sequences into a cell or multi-cellular organism that includes an excisable sequence element that is in turn bounded on either side by specific excision sequences, a repressible promoter operably linked to a gene encoding a site specific recombinase capable of recognizing the specific excision sequences, and a gene operably encoding a repressor specific for the repressible promoter. According to the invention, rendering the transcripts encoding the repressor protein as a template for template directed polymerization of nucleic acids, e.g. in response to some condition, reduces the expression of the repressor protein, thereby derepressing expression of the recombinase, thereby causing excision of the excisable sequence, thus altering the sequence of the DNA molecule in which the excisable sequence was previously found. In this manner, polymerase mediated inhibition of the protein coding activity of a preselected mRNA can be used to induce a gentotypic change in a cell. The presence or absence of the genotypic change in the cell or within a population of such cells can be detected by standard methods, for example, PCR amplification. The resulting genotypic changes present in the cell and its progeny, if any, serve as a record of exposure to the condition that caused the genotypic change to occur, for example, infection of the cell by a particular virus. Thus, a variation of this embodiment of the invention provides another sort of diagnostic assay for viral infection.

Another related embodiment provides introducing a series of sequences into a cell or multi-cellular organism that includes a transiently-active promoter linked to a preselected gene, the promoter and preselected gene being separated by a blocking sequence that is in turn bounded on either side by specific excision sequences, a repressible promoter operably linked to a gene encoding a site specific recombinase capable of recognizing the specific excision sequences, and a gene operably encoding a repressor specific for the repressible promoter. According to the invention, rendering the transcripts encoding the repressor protein as a template for template directed polymerization of nucleic acids, in response to some condition, e.g., provision of a viral polymerase by an infecting virus, reduces the expression of the repressor protein, thereby derepressing expression of the recombinase, thereby causing excision of the blocking sequence, thus providing for expression of the preselected gene. In this manner, polymerase mediated inhibition of the protein coding activity of an mRNA can be used to induce the expression of a preselected gene. In one such embodiment of the invention, the organism is a plant, the repressor protein is inhibited in response to provision of a viral polymerase and the preselected gene is, for example, a lethal/toxin gene or a reporter gene.

The CRE-LOX recombinase excision system, as disclosed for example in U.S. Pat. No. 5,723,765, has been employed for the purpose of selective recombination in numerous systems including plants and animals including mammals and their cells and is readily employed according to the present invention.

As further discussed herein, when the condition triggering the polymerase-mediated inactivation of the repressor is the expression of a preselected RNA transcript (or any RNA transcript having a preselected sequence or sequence region) in the cells of a multicellular organism according to normal developmental regulation, the recombinase derepression embodiment of the invention can be used to detect expression of the preselected RNA transcript generally and to trace the lineage of expression of the preselected RNA transcript, both visibly, in real time, for example by using GFP or a derivative as a recombinase-derepressible gene, or by hybridization probing and/or histologically staining. In a further related embodiment, the derepression is performed on preselected cells in a developing embryo or tissue, for example by microinjection of a primer molecule into the cell(s) or other molecule capable of triggering the polymerase mediated inhibition of the repressor mRNA, in order to track and trace the behavior and/or lineage of the cells and their progeny. The promoter sequence that drives the expression of the preselected gene may be a constitutive promoter or a developmentally-regulated or tissue-specific promoter.

The repressor may also be a regulatory polynucleic acid that directly inhibits the activity, of a second polynucleic acid. For example, the repressor may be a catalytic polynucleic acid, such as a ribozyme or DNAzyme, engineered to cleave or otherwise degrade an mRNA transcript that encodes a peptide having, e.g., reporter activity or gene regulatory activity or cell phenotype altering activity of any sort. Here again, the cell is genetically modified so that the polynucleic acid repressor is operably linked to the activity of a viral nucleic acid polymerase, the functional interaction between the polynucleic acid molecule and the polymerase being regulated by entry of the virus into the cell.

When used to de-repress reporter genes, derepression embodiments of the invention can also be used to provide viral infection detection systems in plants and animals as described above. When used to de-repress suicide genes, e.g., lethal/toxin genes, these derepression embodiments of the invention can also be use to provide viral infection-limiting cell death in plants and animals as described above. For example, in plant embodiments of the invention lethal nucleases such as Bamase and ribonuclease A and 2 can be used and catalytic lethal proteins such as diptheria toxin and ribosomal inhibitor proteins (RIP) can be used. Use of a ribosomal inhibitor protein (“RIP”) gene as a lethal gene for plants, e.g., the saponin 6 RIP, (GenBank ID SOSAPG, Accession No. X15655), is advantageous since RIP directly interferes in the expression of all protein in a plant cell, without being toxic to other organisms which ingest a plant having such cells.

Lethal/toxin genes applicable for animal cell embodiments of the invention include, but are not limited to: (a) apoptosis inducing tumor suppressor genes (e.g., p53), (b) cytotoxic genes (e.g., tumor necrosis factor, interferon-alpha), (c) suicide genes (e.g., cytosine deaminase, thymidine kinase), and (d) toxins such as pseudomonas endotoxin, ricin or diphtheria toxin subunits.

As a further example, genes coding for enzymes that convert a protoxin to a toxin can be used as the genes that are de-repressible according to the derepression embodiment. In this manner, cell death is made conditional on both the derepression of the converting enzyme and application of the protoxin to the cells/organism.

The invention is not limited to the type of cell or multi-cellular organism in which the invention is embodied or implemented. Further, cells and organisms in which the invention is embodied or implemented are within the scope of the invention. The genetic modification of cells and organisms, including complex organisms, to embody the invention, can be performed using any of the standard methods known in the art.

One embodiment of the invention comprises genetically modifying a cell or multi-cellular organism cell to render it capable of utilizing at least one preselected cell-encoded or viral polynucleic acid as a template for the template-directed polymerization of polynucleic acids by a template-directed polynucleic acid polymerase so that the activity of the polynucleic acid molecule is inhibited, wherein the cell or multi-cellular organism not so modified is at least substantially incapable of inhibiting the activity of the polynucleic molecule by utilizing it as a template for template-directed nucleic acid polymerization by a polynucleic acid polymerase. In one example of this embodiment, resistance to viral infection can be imparted to the cell or multi-cellular organism when the target polynucleic acid molecule is a viral genomic polynucleic molecule, viral replication intermediate polynucleic acid molecule, viral mRNA transcript, or other viral polynucleic acid molecule, the activity of which is required for, or contributes to, viral pathogenicity.

A further embodiment of the invention is directed to the above embodiment wherein the cell or multi-cellular organism not so genetically modified is at least substantially incapable of inhibiting the activity of the polynucleic molecule by utilizing it as a template for template-directed nucleic acid polymerization by a polynucleic acid polymerase, even upon providing the cell or multi-cellular organism, by genetic modification or otherwise, with putative primer molecules having a 3′ region of complementarity to the polynucleic acid molecule.

A still further related embodiment of the invention comprises, genetically modifying a cell or multi-cellular organism to express a template-directed polynucleic acid polymerase capable of utilizing the polynucleic acid as a template so that its activity is inhibited.

Another related embodiment further comprises genetically modifying the cell or multi-cellular organism to produce a polynucleic acid primer molecule capable of directing a template directed polynucleic acid polymerase not endogenous, i.e., not normally coded for or expressed by the cell, to utilize the polynucleic acid as a template so that its activity is inhibited. Such a polymerase includes, for example, one for which the cell is genetically engineered to express or that is provided by a virus as a result of infection of the cell by the virus.

A still further related embodiment of the invention comprises genetically modifying a cell or multi-cellular organism to express a preselected nuclease capable of degrading a target polynucleic acid molecule which has been rendered a template for a polymerase, during or after template directed nucleic acid polymerization by the polymerase, wherein the cell or multi-cellular organism otherwise lacks such a nuclease or wherein the efficiency of the degradation is increased by genetically modifying the cell or multi-cellular organism to express the pre-selected nuclease.

Another embodiment of the invention comprises providing the cell or multi-cellular organism with a template-directed polynucleic acid polymerase having an integral nuclease capable of degrading a preselected polynucleic acid molecule which serves as a template for the polymerase, during or after template directed nucleic acid polymerization by the polymerase, so that the activity of the preselected polynucleic acid molecule is inhibited.

### EXAMPLES OF TYPES OF VIRUSES FOR WHICH THE EMBODIMENTS OF THE INVENTION ARE GENERALLY APPLICABLE

Tables 1-10 illustrate various categories of viruses and virus-like polynucleic acid molecules for which the diagnostic and viral resistance embodiments of the invention can be employed. Template-directed polymerases of, or used by, these viruses are examples of polymerases that can be used for polymerase-mediated gene regulation according to the invention. Each of the tables shows the order, family [subfamily] and genus of viruses for which the present invention can be employed. In addition, at least one type species example is provided for each genus listed. Tables 1-10 are arranged according to the following categories: Table 1 -dsDNA viruses; Table 2—ssDNA viruses; Table 3—DNA reverse transcribing viruses; Table 4—RNA reverse transcribing viruses; Table 5—dsRNA Viruses; Table 6—negative-sense ssRNA viruses; Table 7—positive-sense ssRNA viruses; Table 8—naked RNA viruses; Table 9—viroids; and Table 10—subviral agents.

### EXAMPLES OF PLANTS AND THEIR VIRUSES FOR WHICH THE EMBODIMENTS OF THE INVENTION ARE APPLICABLE

The following list illustrates examples of various crop plants and their respective common viruses for which the diagnostic and viral resistance methods of the invention can be embodied: alfalfa—alfalfa mosaic, lucerene transient streak, alfalfa latent; barley—barley stripe, barley yellow dwarf, barley yellow streak, barley yellow streak mosaic, brome mosaic, oat blue dwarf; bean—bean common mosaic virus, bean yellow mosaic virus, beet curly top, cucumber mosaic virus, pea enation mosaic; beet-beet cryptic virus 1, beet cryptic virus 2, beet cryptic virus 3, beet curly top, beet mosaic, beet necrotic yellow vein, beet pseudoyellows, beet soilborne mosaic, beet western yellows, beet yellows, brassicas—cauliflower mosaic virus, turnip mosaic; capsicum species—alfalfa mosaic, beet curly top, cucumber mosaic, potato virus x, potato virus y, tobacco etch, tobacco mosaic, tobacco rattle, tomato spotted wilt; carrots—alfalfa mosaic, carrot mottle, carrot red leaf, carrot thin leaf, carrot mottley dwarf; chrysanthemum species—chrysanthemum aspermy, chrysanthemum mosaic, chrysanthemum virus b, tomato aspermy, impatiens necrotic spot; corn—maize dwarf, maize chlorotic dwarf, maize chlorotic mottle, maize dwarf, maize raydo fino, maize stripe, maize white line mosaic; cotton—cotton leaf crumple; cucumis species—alfalfa mosaic, beet curly top, cucumber mosaic virus, lettuce infectious yellows, papaya ringspot virus, tomato spotted wilt, watermelon mosaic virus 2, squash mosaic virus, zucchini yellow mosaic; cucurbita species—beet curly top, cucumber mosaic virus, papaya ringspot virus, watermelon mosaic virus 2, squash mosaic virus, squash leaf curl, tomato spotted wilt; gladiolus-bean common mosaic virus, lily symptomless virus; lettuce—alfalfa mosaic, beet western yellows, cucumber mosaic, sowthisltle yellow vein, tobacco rattle, tobacco ringspot, tomato spotted wilt, turnip mosaic; papaya—papaya ringspot virus; pea—bean leaf roll, bean yellow mosaic virus, pea enation mosaic virus, tomato spotted wilt, pea seedborne mosaic, pea streak; peanut—peanut mottle, peanut stripe, peanut stunt, tomato spotted wilt; pepper—cucumber mosaic virus, papaya ringspot virus, watermelon mosaic virus 2, squash mosaic virus, pepper cryptic virus 1, pepper mild mottle, pepper mottle, pepper veinal mottle; potatoes—potato leaf roll, potato virus y, potato virus x, potato virus a, potato virus m, potato virus s, tobacco rattle, tomato spotted wilt; rice—rice hoja blanca; sorghum—maize dwarf mosaic, sugarcane mosaic, maize chlorotic dwarf; soybean—bean pod mottle, cowpea chlorotic mottle, peanut mottle, soybean dwarf, soybean mosaic, tobacco ringspot, tobacco streak, bean yellow mosaic, cowpea severe, peanut stripe, tobacco mosaic; strawberry—tomato ringspot, strawberry chlorosis, strawberry crinkle, strawberry latent, strawberry mottle, strawberry vein banding; sugar beets—beet curly top, beet cryptic virus 1, beet cryptic virus 2, beet crytic virus 3, beet mosaic, beet necrotic yellow vein, beet pseudoyellows, beet soilbome mosaic, beet western yellows, beet yellows; sweet potato—sweet potato feathery mottle; tobacco—tobacco mosaic, potato virus y, tomato spotted wilt, tobacco etch, tobacco vein mottling, alfalfa mosaic; tomatoes—alfalfa mosaic, cucumber mosaic, beet curly top, tobacco etch, potato virus y, tomato bushy stunt, tomato mosaic, tomato spotted wilt, tomato ringspot, tomato mottle; and wheat—agropyron mosaic, barley stripe, barley yellow dwarf, barley yellow streak, barley yellow streak mosaic.

### EXAMPLES OF ANIMALS (OR THE CELLS THEREOF) AND THEIR VIRUSES FOR WHICH THE EMBODIMENTS OF THE INVENTION ARE APPLICABLE

Viral pathogenic infection of agricultural animals poses a significant problem for animal health and food production. The present invention provides methods for producing transgenic livestock and other transgenic agricultural animals that are resistant to prespecified viral pathogens. Accordingly, diagnostic and viral resistance embodiments of the invention can be employed for agricultural animals such as, but not limited to, bovids (cattle; sheep; goats, etc.), swine, fowl (chicken; quail; turkey, duck, goose, etc.); fishes, crustaceans (shrimp, crayfish, lobster, crab, etc.) and mollusca (oyster, mussel, clam, etc.).

Tables 11A and 11B show examples of various animals and their common viral pathogenic diseases for which the diagnostic and viral resistance methods of the invention can be embodied.

Aquacultured (maricultured) species have heretofore been particularly susceptible to rapid and dramatic loss as a result of viral pathogens, in part due to the typically high density of the culture conditions.

Fish viruses for which the embodiments of the invention are applicable include, but are not limited to: rhabdoviruses such as spring viremeia of carp virus; bimaviridae infections such as pancreatic necrosis virus; iridoviridae such as fish lymphocytes disease virus; salmonids with infectious hematopoietic necrocosis (IHN); viral hemorrhagic septicemia (VHS) virus; marine viral haemorrhagic septicaemia (VHS), a disease closely related to VHS known from freshwater rainbow trout farming; largemouth bass virus (LMBV); lymphocystis, a viral disease in common dab; infectious salmon anemia; Koi Herpes Virus (KHV); and Monodon baculo virus (MBV).

Shrimp viruses for which the embodiments of the invention are applicable include, but are not limited to, Infectious Hyposdermal and Hematopoietic Necrosis Virus (IHHNV); Taura Syndrome Virus (TSV); White Spot Syndrome Virus (WSSV); and Yellow Head Virus (YHV).

The genetic modification of animals to impart viral resistance against preselected viruses can also provide benefits for human health in the case of viral zoonoses, i.e., viral diseases that are transmitted to humans from animals. For example, it is well established that swine and avians, e.g., ducks, are hosts to influenza viruses and transmit such viruses to each other and to humans. Other viral pathogens are transmissible to humans from insects. According to one embodiment of the invention, genetically modified pigs embodying resistance to influenza virus according to the invention are raised, thereby reducing zoonetic transmission of the disease to the human population.

### EMBODIMENT RELATED TO TRANSPLANATION OF CELLS GENETICALLY MODIFIED TO EMBODY THE INVENTION

One embodiment of the invention provides a method of making a subject mammal resistant to pathogencity of a preselected viral pathogen to which the organism is ordinarily susceptible, comprising the steps of: providing genetically-modified cells capable of integrating with a tissue of the subject mammal, the tissue ordinarily being susceptible to the viral pathogen, wherein (i) the modification causes the activity of at least one preselected viral polynucleic acid molecule of the virus to be diminished in the cells as a result of the preselected viral polynucleic acid molecule being rendered a template for the template directed polymerization of nucleic acid molecules within the cell; or (ii) the modification causes the activity of at least one preselected cellular RNA transcript to be diminished in response to infection by the virus as a result of the preselected cellular transcript being rendered a template for the template directed polymerization of nucleic acid molecules within the cell, resulting in (a) resistance of the cell and/or (b) resistance of the multi-cellular organism as a whole to the pathogenicity of the virus by reducing the replication and/or spread of the virus within the subject; and introducing the genetically modified cells into the subject mammal.

A related embodiment of the invention provides that the mammal is already infected with the virus when the genetically modified cells are introduced into the mammal.

A further, related embodiment provides that the step of providing the genetically modified cells further comprises first isolating unmodified cells from the tissue of the subject mammal, then modifying these cells as described, and thereafter introducing the so-modified cells back into the subject mammal.

Further embodiments of the above-described methods provide that the subject mammal is a human being and (i) the tissue comprises lymphoid cells or lymphoid cell precursors and the virus is a human immunodeficiency virus, such as HIV-1 or HIV-2; or (ii) the tissue comprises hepatic cells or hepatic cell precursors and the virus is a Hepatitis virus, for example, Hepatitis B or Hepatitis C.

Another related embodiment of the invention provides genetically-modified cells capable of integrating with a tissue of a subject mammal, the tissue ordinarily being susceptible to a viral pathogen, wherein (i) the modification causes the activity of at least one preselected viral polynucleic acid molecule of the virus to be diminished in the cells as a result of the preselected viral polynucleic acid being rendered a template for the template directed polymerization of nucleic acid molecules within the cell; or (ii) the modification causes the activity of at least one preselected cellular RNA transcript to be diminished in response to infection by the virus as a result of the preselected cellular transcript being rendered a template for the template directed polymerization of nucleic acid molecules within the cell, resulting in (a) resistance of the cell to the virus and/or (b) resistance of the mammal as a whole to the pathogenicity of the virus by reducing the replication and/or spread of the virus within the subject upon integration of the genetically modified cells into the subject mammal.

### RNA SILENCING

In a first variation of the cell-based and multi-cellular organism-based embodiments of the present invention, the cells constituting the elements of the invention are not capable of RNA silencing in response to long dsRNA or long RNA/cDNA hybrid molecules and the rendering of a preselected RNA molecule to be a template for a preselected polynucleic acid polymerase to reduce the “normal” activity of the polynucleic acid molecule forms a long dsRNA molecule or long RNA/cDNA hybrid which does not trigger RNA silencing of polynucleic acid molecules in the cell.

In a related variation of the invention, the cells constituting the elements of the invention are not capable of RNA silencing in response to long dsRNA or long RNA/cDNA hybrid molecules but are capable of RNA silencing in response to siRNAs or similar small RNA/cDNA hybrid molecules. Various mammalian cells including human cells have this characteristic. In a related subvariation of the invention, such cells are made to express a member of the RNAse III family, such as Dicer, to cause the long dsRNA or long RNA/cDNA hybrid molecules formed by rendering the preselected RNA molecule to be a template for a preselected polynucleic acid polymerase to reduce the activity of the polynucleic acid molecule to be processed into siRNAs or small RNA/cDNA hybrids which trigger RNA silencing of polynucleic acid molecules in the cell.

In a further variation of the invention, the cells constituting the elements of the invention are incapable of RNA silencing in response to long dsRNA or long RNA/cDNA hybrid molecules, whether or not such molecules are processed by an RNAse III enzyme, and the rendering of a preselected RNA molecule to be a template for a preselected polynucleic acid polymerase to reduce the activity of the preselected polynucleic acid molecule forms such a long dsRNA molecule or long RNA/cDNA hybrid. In one embodiment of this variation, an RNAse III enzyme is expressed in the cell(s), for example, by genetically-modifying the cell(s) to express the enzyme, and the RNAse III processes the long dsRNA molecule or long RNA/cDNA hybrid but RNA silencing is not triggered in response. In accordance with the described role of RISC in RNA silencing, such a cell can be created by genetically modifying an otherwise RNAi-RNA silencing-competent cell to knock out, for example by homologous recombination, one or more components of the RISC complex.

In another variation of the invention, the cells constituting the elements of the invention are capable of RNA silencing in response to long dsRNA or long RNA/cDNA hybrid molecules and the rendering of a preselected RNA molecule to be a template for a preselected polynucleic acid polymerase, to reduce the activity of the preselected polynucleic acid molecule, forms such a long dsRNA molecule or long RNA/cDNA hybrid which fturther triggers RNA silencing of polynucleic acid molecules in the cell.

When RNA silencing is triggered by rendering the preselected polynucleic acid molecule(s) a template in the cells embodying the invention, the polynucleic acid molecules silenced may be other copies of the same preselected polynucleic acid molecule which may be present in the cell, similar polynucleic acid molecules encoded by the same gene or different polynucleic acid molecules encoded by different genes but which have regions of complementarity with at least one strand of the siRNA fragment(s) generated from the preselected polynucleic acid molecule which is rendered as a template.

In a related variation of the invention, the cells constituting the elements of the invention are capable of RNA silencing in response to long dsRNA but not in response to long RNA/cDNA hybrid molecules. In this variation, use of a reverse transcriptase to render a preselected RNA molecule to be template for a preselected polynucleic acid polymerase to reduce the activity of the preselected polynucleic acid molecule creates a long RNA/cDNA hybrid which does not trigger said RNA silencing, while use of a RNA-dependent RNA polymerase creates a long dsRNA which does trigger said RNA silencing.

In another related variation, the cells constituting the elements of the invention are capable of RNA silencing in response to long RNA/cDNA hybrids but a reverse transcriptase having an integral or closely-associated RNAse H is used as the polymerase which renders the preselected polynucleic acid molecule as a template for template directed polymerization to reduce the activity of the preselected polynucleic acid molecule so that a long RNA/cDNA hybrid is not formed as a result of said polymerization. In a similar related variation, the RNAse H is not integral or closely associated with the reverse transcriptase, but is nevertheless expressed in the cell and active against the RNA template component of the RNA/cDNA hybrid being formed by the reverse trascriptase. In either case, the RNAse H hydrolyzes the RNA template strand as the cDNA is synthesized so that formation of a long RNA/cDNA hybrid molecule competent for RNA silencing is at least substantially not formed and RNA silencing is at least substantially prevented.

One RNA silencing related embodiment of the invention provides introducing a series of sequences into a cell or multi-cellular organism that includes an excisable sequence element that is in turn bounded on either side by specific excision sequences, a repressible promoter operably linked to a gene encoding a site specific recombinase capable of recognizing the specific excision sequences, and a gene operably encoding a repressor specific for the repressible promoter. According to the invention, selectively triggering an RNA silencing cascade against the repressor, e.g., in response to some condition, reduces the expression of the repressor protein, thereby derepressing expression of the recombinase, thereby causing excision of the excisable sequence, thus altering the sequence of the DNA molecule in which the excisable sequence was previously found. In this manner, RNA silencing of the protein coding activity of a preselected mRNA can be used to induce a genotypic change in a cell. The presence or absence of the genotypic change in the cell or within a population of such cells can be detected by standard molecular biological methods. The resulting genotypic changes present in the cell and its progeny if any can also serve as a record of exposure to the condition which caused the genotypic change to occur, for example, infection of the cell by particular virus(es) or the presence of any preselected RNA sequence in the cell. Thus a variation of this embodiment of the invention provides another sort of diagnostic assay for viral infection.

In a related embodiment of the invention, the excisable sequence element comprises an expression cassette comprising at least one gene of interest, and transcriptional regulatory elements providing for expression of the gene in a constitutive or regulated manner. In this embodiment, when recombinase expression is de-repressed, as described above, the excisable element is deleted and expression of the at least one gene of interest from the cassette is eliminated or the possibility of regulative expression of the gene is eliminated as a result of deleting the cassette.

Another RNA-silencing related embodiment provides introducing a series of sequences into a cell or multi-cellular organism that includes a transiently-active promoter linked to a preselected gene the promoter and preselected gene being separated by a blocking sequence that is in turn bounded on either side by specific excision sequences, a repressible promoter operably linked to a gene encoding a site specific recombinase capable-of recognizing the specific excision sequences, and a gene operably encoding a repressor specific for the repressible promoter. According to the invention, selectively triggering an RNA cascade against the repressor protein, e.g., in response to some condition, renders the transcripts encoding the repressor protein as a template for template directed polymerization of nucleic acids, in response to some condition, e.g., provision of a viral polymerase by an infecting virus, and reduces the expression of the repressor protein, thereby derepressing expression of the recombinase, thereby causing excision of the blocking sequence, thus providing for expression of the preselected gene. In this manner, inactivation of the protein coding activity of an mRNA via an RNA silencing cascade can be used to induce the expression of a preselected gene. In one such embodiment of the invention, the organism is a plant, the repressor protein is inhibited in response to viral infection and the preselected gene is, for example, a lethal/toxin gene or a reporter gene or an antiviral gene, for example, a gene coding for RNA silencing-inducing short RNA hairpins specific for the inhibition of viral polynucleic acid molecules or cellular polynucleic acid molecules in the cell.

According to the embodiments of the invention in which an mRNA transcript, coding for a transcriptional regulatory protein, is targeted by RNA silencing in response to some stimuli, the stimuli may be of any sort including, but not limited to, the following cases:

the stimuli comprises (i) contacting the cell with a prepared, e.g., synthetic, siRNA at least one of the strands of the siRNA being at least substantially complementary to the mRNA transcript or (ii) providing the cell with expression of such an siRNA;

the stimuli comprises contacting the cell with a short RNA hairpin molecule wherein at least one of the complementary segments of the double stranded stem structure is at least substantially complementary to the mRNA transcript, or providing the cell with expression of such a molecule

the stimuli comprises providing the cell with long dsRNA which is processed into siRNAs, the siRNAs having at least one strand having at least substantial complementarity with the mRNA molecule;

the stimuli comprises infection of the cell by a virus and the virus characteristically forms long dsRNA which triggers RNA silencing in response to the infection, which RNA silencing comprises production of siRNAs wherein at least one strand is at least substantially complementary to the mRNA transcript;

the stimuli comprises infection ofthe cell by a virus and the cell is genetically modified to render an mRNA transcript of the virus or another polynucleic acid molecule of the virus as a template for template directed polymerization of nucleic acids so that a long dsRNA or long RNA/cDNA is formed, the long molecule triggering RNA silencing in response to the infection, which RNA silencing comprises production of siRNAs wherein at least one strand is at least substantially complementary to the mRNA transcript;

the stimuli comprises rendering the mRNA molecule initially as a template for the template directed polymerization of nucleic acids by a polymerase, wherein siRNAs or small RNA/cDNA hybrids are produced from the double stranded polymerization product, said siRNAs or small RNA/cDNA hybrids causing RNA silencing of other of the same mRNA molecules coding for the regulatory protein;

the stimuli comprises expression of an RNA transcript, e.g., an mRNA transcript under control of developmentally regulated promoters and/or other transcriptional regulatory elements, (i) the cell being capable (genetically modified if necessary) of rendering the transcript as a nucleic acid polymerization template to form a long dsRNA or long RNA/cDNA molecule which triggers RNA silencing, or (ii) the cell expressing a long single stranded RNA or DNA molecule at least substantially complementary to the RNA transcript, wherein, upon expression of the RNA transcript, the transcript and the expressed complementary molecule hybridize to form an at least partially double stranded molecule capable of triggering RNA silencing or (3) the RNA transcript being itself capable of triggering RNA silencing, for example, the transcript being or comprising an RNA silencing-competent RNA hairpin.

In the above examples, when the sequence of at least a part of the long dsRNA or long RNA/cDNA molecule is known (such as when the trigger is a known virus or cellular transcript), the gene coding for the transcriptional regulatory protein, can, if necessary, be modified to contain regions of homology to one or both of the strands of the long dsRNA or long RNA/cDNA molecule so that the siRNA in conjunction with RISC will target these regions using siRNAs formed from the long double stranded molecules. Conversely, if the polynucleic acid molecule for which it is desired to trigger RNA silencing upon expression of the molecule is encoded by a cellular gene (endogenous or transgene), the sequence of the gene for this molecule can, if necessary, be modified to comprise regions of homology to the mRNA transcript for the regulatory protein so that upon the polynucleic acid molecule becoming double stranded it is processed to render siRNAs wherein at least one strand is at least substantially complementary to the target repressor protein transcript.

When the siRNA in conjunction with RISC functions to prime the target molecule and induce template directed polymerization of nucleic acids using the target as a template, the introduction of the regions of homology is an introduction of a PBS according to the invention and, likewise, providing an siRNA is an example of providing a primer molecule.

The invention also comprises transitive siRNA embodiments. Those skilled in the art will readily appreciate that at least one intermediate polynucleic acid molecule may be used to indirectly mediate the transfer of the RNA silencing signal, via siRNAs, from an initial, or otherwise “upstream”, double-stranded, long triggering molecule to the target mRNA transcript of the transcriptional regulatory protein. In this case the intermediate molecule will have complementary sequences to at least one siRNA strand produced from the initial molecule, thereby rendering it a target for siRNAs formed from the initial molecule, and at least one other sequence, generally in the three-prime direction with respect to the primer strand of said sikNA, the other sequence being complementary or having homology to the ultimate target RNA transcript in question so that formation of a long dsRNA from the intermediate molecule causes the formation of siRNAs wherein at least one strand is complementary to the ultimate target. Thus, when it is known that a first molecule will be subject to RNA silencing, the silencing event can be transitively targeted to one or more selected other molecules by providing the cell with expression of one or more intermediate molecules.

Generally, any first molecule that will be targeted by RNA silencing to produce siRNAs can be modified to induce RNA silencing against a selected downstream molecule by modifying the first molecule (e.g. by modifying the gene expressing the molecule) to comprise sequence that will form siRNA strands having homology with or being complementary to the sequence of the selected downstream molecule. Alternatively, or in addition, a selected molecule can be rendered as a downstream target of the first molecule by modifying the second molecule (e.g. by modifying the gene expressing the molecule) so that it comprises at least one sequence element having homology with or being complementary to the sequence of the first molecule.

The invention also encompasses the case where the siRNA-RISC machinery is not capable of inducing template directed polymerization of nucleic acid molecules but nevertheless binds to the target and optionally causes nuclease activity against the target.

As further discussed herein, when the condition triggering the polymerase-mediated inactivation of the repressor is the expression of a preselected RNA transcript (or any RNA transcript having a preselected sequence) in the cells of a multi-cellular organism according to normal developmental regulation, the recombinase derepression embodiment of the invention can be used to detect expression of the preselected RNA transcript generally and to trace the lineage of expression of the preselected RNA transcript, both visibly, in real time, for example by using GFP or a derivative as a recombinase-derepressible gene, or by histological analysis.

The techniques used for producing genetically-engineered cells and transgenic multi-cellular organisms according to the invention are routine in the art and are, accordingly, only briefly described herein.

As referred to herein, a genetically modified cell or multi-cellular organism refers to a cell or organism that has been genetically engineered to embody genetic sequences required for the functioning of the invention, or which are derived from such a cell or organism and comprise the introduced change(s). Genetically modified cells and genetically-modified organisms according to the invention include cells and organisms genetically engineered to contain transgenes and/or to have genetic sequence alterations including deletions, or which are derived from such a cell or organism and comprise the introduced change(s). Cells transiently transformed with DNA or RNA constructs are also within the scope of the invention.

A variety of routine methods for introducing DNA into cells for the introduction of transgenes to the cells and/or to effectuate other genetic changes in the cells are well established Predetermined deletions and other sequence changes of preselected genes and other genetic sequences in a cell can be performed using homologous recombination techniques as known in the art.

Transgenic animals are animals having cells that contain a transgene, wherein the transgene was introduced into the animal or an ancestor of the animal at a prenatal, e.g., an embryonic, stage. Non-human animals into which transgenes or other discrete genetic changes can be introduced by genetic engineering techniques known in the art include, but are not limited to, mice, goats, sheep, pigs, cows and other domestic farm animals, as well as fish, birds, and crustaceans and insects. A transgenic animal can be created, for example, by introducing a nucleic acid sequence encoding a protein of interest (typically linked to appropriate regulatory elements, such as a constitutive or tissue-specific promoter and/or other regulatory elements) into the male pronuclei of a fertilized oocyte, e.g., by microinjection, and allowing the oocyte to develop in a pseudopregnant female foster animal. An alternative method comprises introducing a desired DNA into the genome an embryonic stem cell and regenerating the organism by introducing the modified stem cell into a developing early-stage embryo. A transgenic founder animal can be used to breed additional animals carrying the transgene.

Techniques for transforming a wide variety of higher plant species are also well established and described in the technical and scientific literature. A DNA sequence coding for a desired gene product can be combined with transcriptional and translational initiation regulatory sequences that will direct the transcription of the sequence from the gene in the intended tissues of the transformed plant.

For example, for overexpression, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′- promoter derived from T-DNA of Agrobacterium tumefaciens, and other transcription initiation regions from various plant genes known to those of skill.

Alternatively, the plant promoter may direct expression of a nucleic acid of the invention in a specific tissue, organ or cell type (i.e. tissue-specific promoters) or may be otherwise under more precise environmental or developmental control (i.e. inducible promoters). Examples of environmental conditions that may effect transcription by inducible promoters include anaerobic conditions, elevated temperature, the presence of light, or application of chemicals such as hormones. Exemplary promoters for this purpose include promoters from glucocorticoid receptor genes (Aoyama and Chua, Plant J 11:605-12 (1997)). Tissue-specific promoters may only promote transcription within a certain time frame of developmental stage within that tissue. Other tissue specific promoters may be active throughout the life cycle of a particular tissue.

Techniques for the production of transgenic plants are well established. For example, the DNA construct comprising a transgene may be introduced directly into the genomic DNA of the plant cell using techniques such as electroporation and microinjection of plant cell protoplasts,.or the DNA constructs can be introduced directly to plant tissue using ballistic methods, such as DNA particle bombardment, or Agrobacterium tumefaciens-mediated transformation techniques, including disarming and use of binary vectors, are well described in the scientific literature. Transformed plant cells that are derived by any of the above transformation techniques can be cultured to regenerate a whole plant that possesses the transformed genotype and thus the desired phenotype such as increased seed mass. Regeneration can also be obtained from plant callus, explants, organs, or parts thereof.

It is to be understood that the above descriptions are meant to be illustrative and not limiting. With respect to its cell-based embodiments, the invention is not limited by the subcellular compartment(s) in which its components are processed and ultimately function. Many embodiments will be apparent to those of skill in the art upon reviewing the above descriptions. The scope of the invention should, therefore, be determined with reference to the appended claims, along with the full scope of equivalents to which such claims are entitled.

TABLE 1
dsDNA Viruses

| Order | Family [Subfamily] | Genus | Type Species Example |
|---|---|---|---|
| Caudovirales | Myoviridae | “T4-like viruses” | Enterobacteria phage T4 |
| | | “P1-like viruses” | Enterobacteria phage |
| | | | P1 |
| | | “P2-like viruses” | Enterobacteria phage |
| | | | P2 |
| | | “Mu-like viruses” | Enterobacteria phage |
| | | | Mu |
| | | “SPO1-like viruses” | Bacillus phage SPO1 |
| | | “ΦH-like viruses” | Halobacterium virus |
| | | | ΦH |
| | Siphoviridae | “λ-like viruses” | Enterobacteria phage λ |
| | | “T1-like viruses” | Enterobacteria phage |
| | | | T1 |
| | | “T5-like viruses” | Enterobacteria phage |
| | | | T5 |
| | | “c2-like viruses” | Lactococcus phage |
| | | | c2 |
| | | “L5-like viruses” | Mycobacterium |
| | | | phage L5 |
| | | “ΨM1-like viruses” | Methanobacterium |
| | | | ΨM1 |
| | Podoviridae | “T7-like viruses” | Enterobacteria phage |
| | | | T7 |
| | | “φ29-like viruses” | Bacillus phage φ29 |
| | | “P22-like viruses” | Enterobacteria phage |
| | | | P22 |
| | Rudiviridae | Rudivirus | Sulfolobus virus |
| | | | SIRV1 |
| | Tectiviridae | Tectivirus | Enterobacteria phage |
| | | | PRD1 |
| | Corticoviridae | Corticovirus | Alteromonas phage |
| | | | PM2 |
| | Lipothrixviridae | Lipothrixvirus | Thermoproteus virus 1 |
| | Plasmaviridae | Plasmavirus | Acholeplasma phage |
| | | | L2 |
| | Fuselloviridae | Fusellovirus | Sulfolobus virus |
| | | | SSV1 |
| | Phycodnaviridae | Chlorovirus | Paramecium bursaria |
| | | | Chlorella virus 1 |
| | | Prasinovirus | Micromonas pusilla |
| | | | virus SP′1 |
| | | Prymnesiovirus | Chrysochromulina |
| | | | brevifilum virus |
| | | | PW1 |
| | | Phaeovirus | Ectocarpus siliculosis |
| | | | virus 1 |
| | | “Sulfolobus SNDV- | Sulfolobus virus |
| | | like viruses” | SNDV |
| | Poxviridae | Orthopoxvirus | Vaccinia virus |
| | [Chordopoxvirinae] | | |
| | | Parapoxvirus | Orf virus |
| | | Avipoxvirus | Fowlpox virus |
| | | Capripoxvirus | Sheeppox virus |
| | | Leporipoxvirus | Myxoma virus |
| | | Suipoxvirus | Swinepox virus |
| | | Molluscipoxvirus | Molluscum |
| | | | contagiosum virus |
| | | Yatapoxvirus | Yaba monkey tumor |
| | | | virus |
| | [Entomopoxvirinae] | Entomopoxvirus A | Melolontha |
| | | | melolontha |
| | | | entomopoxvirus |
| | | Entomopoxvirus B | Amsacta moorei |
| | | | entomopoxvirus |
| | | Entomopoxvirus C | Chironomus luridus |
| | | | entomopoxvirus |
| | Iridoviridae | Iridovirus | Invertebrate |
| | | | iridescent virus 6 |
| | | Chloriridovirus | Invertebrate |
| | | | iridescent virus 3 |
| | | Ranavirus | Frog virus 3 |
| | | Lymphocystivirus | Lymphocystis |
| | | | disease virus 1 |
| | Polydnaviridae | Ichnovirus | Campoletis |
| | | | sonorensis ichnovirus |
| | | Bracovirus | Cotesia melanoscela |
| | | | bracovirus |
| | Herpesviridae | Ictalurivirus (was | Ictalurid herpesvirus 1 |
| | | “Ictalurid herpes-like | |
| | | viruses”) | |
| | [Alphaherpesvirinae] | Simplexvirus | Human herpesvirus 1 |
| | | Varicellovirus | Human herpesvirus 3 |
| | | Mardivirus (was | Gallid herpesvirus 2 |
| | | “Marek's disease-like | |
| | | viruses”) | |
| | | Iltovirus (was | Gallid herpesvirus 1 |
| | | “Infectious laryngo- | |
| | | tracheitis-like | |
| | | viruses”) | |
| | [Betaherpesvirinae] | Cytomegalovirus | Human herpesvirus 5 |
| | | Muromegalovirus | Murid herpesvirus1 |
| | | Roseolovirus | Human herpesvirus 6 |
| | [Gammaherpesvirinae] | Lymphocryptovirus | Human herpesvirus 4 |
| | | Rhadinovirus | Saimiriine |
| | | | herpesvirus 2 |
| | Polyomaviridae | Polyomavirus | Simian virus 40 |
| | Papillomaviridae | Papillomavirus | Cottontail rabbit |
| | | | papillomavirus |
| | Adenoviridae | Mastadenovirus | Human adenovirus C |
| | | Aviadenovirus | Fowl adenovirus A |
| | | Atadenovirus | Ovine adenovirus D |
| | | Siadenovirus | Turkey adenovirus B |
| | Ascoviridae | Ascovirus | Spodoptera |
| | | | frugiperda ascovirus |
| | Baculoviridae | Nucleopolyhedrovirus | Autographa |
| | | | californica |
| | | | nucleopolyhedrovirus |
| | | Granulovirus | Cydia pomonella |
| | | | granulovirus |
| | Nimaviridae | Whispovirus | White spot syndrome |
| | | | virus 1 |
| | Asfarviridae | Asfivirus | African swine fever |
| | | | virus |
| | | Rhizidiovirus | Rhizidiomyces virus |

TABLE 2
ssDNA Viruses

| | Family | | Type Species |
|---|---|---|---|
| Order | [Subfamily] | Genus | Example |
| | Inoviridae | Inovirus | Enterobacteria |
| | | | phage M13 |
| | | Plectrovirus | Acholeplasma |
| | | | phage MV-L51 |
| | Microviridae | Microvirus | Enterobacteria |
| | | | phage φX174 |
| | | Spiromicrovirus | Spiroplasma phage 4 |
| | | Bdellomicrovirus | Bdellovibrio phage |
| | | | MAC1 |
| | | Chlamydiamicrovirus | Chlamydia phage 1 |
| | Geminiviridae | Mastrevirus | Maize streak virus |
| | | Curtovirus | Beet curly top virus |
| | | Begomovirus | Bean golden mosaic |
| | | | virus —Puerto Rico |
| | Circoviridae | Circovirus | Porcine circovirus |
| | | Gyrovirus | Chicken anaemia |
| | | | virus |
| | Nanoviridae | Nanovirus | Subterranean clover |
| | | | stunt virus |
| | | Babuvirus | Babana bunchy top |
| | | | virus |
| | Parvoviridae | Parvovirus | Murine Minute |
| | [Parvovirinae] | | virus |
| | | Erythrovirus | B19 virus |
| | | Dependovirus | Adeno-associated |
| | | | virus 2 |
| | [Densovirinae] | Densovirus | Junonia coenia |
| | | | densovirus |
| | | Iteravirus | Bombyx mori |
| | | | densovirus |
| | | Brevidensovirus | Aedes aegypti |
| | | | densovirus |

TABLE 3
DNA Reverse Transcribing Viruses

| | | | Type Species |
|---|---|---|---|
| Order | Family | Genus | Example |
| | Pseudoviridae | Pseudovirus | Saccharomyces |
| | | | cerevisiae Ty1 virus |
| | | Hemivirus | Drosophila |
| | | | melanogaster copia |
| | | | virus |
| | Metaviridae | Metavirus | Saccharomyces |
| | | | cerevisiae Ty3 virus |
| | | Errantivirus | Drosophila |
| | | | melanogaster gypsy |
| | | | virus |
| | Hepadnaviridae | Orthohepadnavirus | Hepatitis B virus |
| | | Avihepadnavirus | Duck hepatitis B |
| | | | virus |
| | Caulimoviridae | Badnavirus | Commelina yellow |
| | | | mottle virus |
| | | Caulimovirus | Cauliflower mosaic |
| | | | virus |
| | | Tungrovirus (was | Rice tungro |
| | | “Rice tungro bacilliform- | bacilliform virus |
| | | like viruses”) | |
| | | Soymovirus (was | Soybean chlorotic |
| | | “Soybean chlorotic | mottle virus |
| | | mottle-like viruses”) | |
| | | Cavemovirus (was | Cassava vein |
| | | “Cassava vein | mosaic virus |
| | | mosaic-like | |
| | | viruses”) | |
| | | Petuvirus (was | “Petunia vein |
| | | “Petunia vein | clearing virus |
| | | clearing-like | |
| | | viruses”) | |

TABLE 4
RNA Reverse Transcribing Viruses

| | Family | | Type Species |
|---|---|---|---|
| Order | [Subfamily] | Genus | Example |
| | Retroviridae | Alpharetrovirus | Avian leucosis virus |
| | [Orthoretrovirinae] | | |
| | | Betaretrovirus | Mouse mammary |
| | | | tumor virus |
| | | Gammaretrovirus | Murine leukeamia |
| | | | virus |
| | | Deltaretrovirus | Bovine leukaemia |
| | | | virus |
| | | Epsilonretrovirus | Walleye dermal |
| | | | sarcoma virus |
| | | Lentivirus | Human |
| | | | immunodeficiency |
| | | | virus 1 |
| | [Spumaretrovirinae] | Spumavirus | Simian foamy virus |

TABLE 5
dsRNA Viruses

| | | | Type Species |
|---|---|---|---|
| Order | Family | Genus | Example |
| | Cystoviridae | Cystovirus | Pseudomonas phage |
| | | | Ψ6 |
| | Reoviridae | Orthoreovirus | Mammalian |
| | | | orthoreovirus |
| | | Orbivirus | Bluetongue virus |
| | | Rotavirus | Rotavirus A |
| | | Coltivirus | Colorado tick fever |
| | | | virus |
| | | Seadornavirus | Kadipiro virus |
| | | Aquareovirus | Aquareovirus A |
| | | Cypovirus | Cypovirus 1 |
| | | Entomoreovirus | Hyposoter exiguae |
| | | | reovirus |
| | | Fijivirus | Fiji disease virus |
| | | Phytoreovirus | Rice dwarf virus |
| | | Oryzavirus | Rice ragged stunt |
| | | | virus |
| | Birnaviridae | Aquabirnavirus | Infectious |
| | | | pancreatic necrosis |
| | | | virus |
| | | Avibirnavirus | Infectious bursal |
| | | | disease virus |
| | | Entomobirnavirus | Drosophila X virus |
| | Totiviridae | Totivirus | Saccharomyces |
| | | | cerevisiae virus L-A |
| | | Giardiavirus | Giardia lamblia |
| | | | virus |
| | | Leishmaniavirus | Leishmania RNA |
| | | | virus 1-1 |
| | Chrysoviridae | Chrysovirus | Penicillium |
| | | | chrysogenum virus |
| | Partitiviridae | Partitivirus | Atkinsonella |
| | | | hypoxylon virus |
| | | Alphacryptovirus | White clover cryptic |
| | | | virus 1 |
| | | Betacryptovirus | White clover cryptic |
| | | | virus 2 |
| | Hypoviridae | Hypovirus | Cryphonectria |
| | | | hypovirus 1-EP713 |
| | | Varicosavirus | Lettuce big-vein |
| | | | virus |

TABLE 6
Negative Sense ssRNA Viruses

| | | | Type Species |
|---|---|---|---|
| Order | Family | Genus | Example |
| Mononegavirales | Paramyxoviridae | Respirovirus | Sendai virus |
| | [Paramyxovirinae] | | |
| | | Morbillivirus | Measles virus |
| | | Rubulavirus | Mumps virus |
| | | Henipavirus | Hendra virus |
| | | Avulavirus | Newcastle disease |
| | | | virus |
| | [Pneumovirinae] | Pneumovirus | Human respiratory |
| | | | syncytial virus |
| | | Metapneumovirus | Avian pneumovirus |
| | Rhabdoviridae | Vesiculovirus | Vesicular stomatitis |
| | | | Indiana virus |
| | | Lyssavirus | Rabies virus |
| | | Ephemerovirus | Bovine ephemeral fever |
| | | | virus |
| | | Cytorhabdovirus | Lettuce necrotic |
| | | | yellows virus |
| | | Nucleorhabdovirus | Potato yellow |
| | | | dwarf virus |
| | | Novirhabdovirus | Infectious |
| | | | hematopoietic |
| | | | necrosis virus |
| Mononegavirales | Filoviridae | Marburgvirus (was | Lake Victoria |
| | | “Marburg-like | marburgvirus (was |
| | | viruses”) | Marburgvirus) |
| | | Ebolavirus (was | Zaire ebolavirus |
| | | “Ebola-like | (was Zaire Ebola |
| | | viruses”) | virus) |
| | Bornaviridae | Bornavirus | Borna disease virus |
| | Orthomyxoviridae | Influenzavirus A | Influenza A virus |
| | | Influenzavirus B | Influenza B virus |
| | | Influenzavirus C | Influenza C virus |
| | | Thogotovirus | Thogoto virus |
| | | Isavirus | Infectious salmon |
| | | | anemia virus |
| | Bunyaviridae | Orthobunyavirus | Bunyamwera virus |
| | | Hantavirus | Hantaan virus |
| | | Nairovirus | Dugbe virus |
| | | Phlebovirus | Rift Valley fever |
| | | | virus |
| | | Tospovirus | Tomato spotted |
| | | | wilt virus |
| | Arenaviridae | Arenavirus | Lymphocytic |
| | | | choriomeningitis |
| | | | virus |
| | | Ophiovirus | Citrus psorosis |
| | | | virus |
| | | Tenuivirus | Rice stripe virus |
| | | Deltavirus | Hepatitis delta |
| | | | virus |

TABLE 7
Positive Sense ssRNA Viruses

| | | | Type Species |
|---|---|---|---|
| Order | Family | Genus | Example |
| | Leviviridae | Levivirus | Enterobacteria phage |
| | | | MS2 |
| | | Allolevivirus | Enterobacteria phage |
| | | | Qβ |
| | Dicistroviridae | Cripavirus | Cricket paralysis |
| | | | virus |
| | | Iflavirus (was | Infectious flacherie |
| | | “Infectious | virus |
| | | flacherie-like | |
| | | viruses”) | |
| | Picornaviridae | Enterovirus | Poliovirus |
| | | Rhinovirus | Human rhinovirus A |
| | | Hepatovirus | Hepatitis A virus |
| | | Cardiovirus | Encephalomyocarditis |
| | | | virus |
| | | Aphthovirus | Foot-and-mouth |
| | | | disease virus |
| | | Parechovirus | Human parechovirus |
| | Sequiviridae | Sequivirus | Parsnip yellow fleck |
| | | | virus |
| | | Waikavirus | Rice tungro spherical |
| | | | virus |
| | Comoviridae | Comovirus | Cowpea mosaic virus |
| | | Fabavirus | Broad bean wilt |
| | | | virus 1 |
| | | Nepovirus | Tobacco ringspot |
| | | | virus |
| | Potyviridae | Potyvirus | Potato virus Y |
| | | Rymovirus | Ryegrass mosaic |
| | | | virus |
| | | Bymovirus | Barley yellow mosaic |
| | | | virus |
| | | Macluravirus | Maclura mosaic virus |
| | | Ipomovirus | Sweet potato mild |
| | | | mottle virus |
| | | Tritimovirus | Wheat streak mosaic |
| | | | virus |
| | Caliciviridae | Vesivirus | Swine vesicular |
| | | | exanthema virus |
| | | Lagovirus | Rabbit hemorrhagic |
| | | | disease virus |
| | | Norovirus (was | Norwalk virus |
| | | “Norwalk-like | |
| | | viruses”) | |
| | | Sapovirus (was | Sapporo virus |
| | | “Sapporo-like | |
| | | viruses”) | |
| | Hepeviridae | Hepevirus (was | Hepatitis E virus |
| | | “Hepatitis E-like | |
| | | viruses”) | |
| | Astroviridae | Astrovirus | Human astrovirus 1 |
| | Nodaviridae | Alphanodavirus | Nodamura virus |
| | | Betanodavirus | Striped jack nervous |
| | | | necrosis virus |
| | Tetraviridae | Betatetravirus | Nudaurelia capensis β |
| | | | virus |
| | | Omegatetravirus | Nudaurelia capensis |
| | | | ω virus |
| | Tombusviridae | Tombusvirus | Tomato bushy stunt |
| | | | virus |
| | | Carmovirus | Carnation mottle |
| | | | virus |
| | | Necrovirus | Tobacco necrosis |
| | | | virus A |
| | | Dianthovirus | Carnation ringspot |
| | | | virus |
| | | Machlomovirus | Maize chlorotic |
| | | | mottle virus |
| | | Avenavirus | Oat chlorotic stunt |
| | | | virus |
| | | Aureusvirus | Pothos latent virus |
| | | Panicovirus | Panicum mosaic virus |
| Nidovirales | Coronaviridae | Coronavirus | Infectious bronchitis |
| | | | virus |
| | | Torovirus | Equine torovirus |
| | Arteriviridae | Arterivirus | Equine arteritis virus |
| | Roniviridae | Okavirus | Gill-associated virus |
| | Togaviridae | Alphavirus | Sindbis virus |
| | | Rubivirus | Rubella virus |
| | Flaviviridae | Flavivirus | Yellow fever virus |
| | | Pestivirus | Bovine viral diarrhea |
| | | | virus |
| | | Hepacivirus | Hepatitis C virus |
| | Bromoviridae | Alfamovirus | Alfalfa mosaic virus |
| | | Ilarvirus | Tobacco streak virus |
| | | Bromovirus | Brome mosaic virus |
| | | Cucumovirus | Cucumber mosaic |
| | | | virus |
| | | Oleavirus | Olive latent virus 2 |
| | Closteroviridae | Closterovirus | Beet yellows virus |
| | | Crinivirus | Lettuce infectious |
| | | | yellows virus |
| | | Ampelovirus | Grapevine leafroll- |
| | | | associated virus 3 |
| | Barnaviridae | Barnavirus | Mushroom |
| | | | bacilliform virus |
| | Luteoviridae | Luteovirus | Barley yellow dwarf |
| | | | virus-PAV |
| | | Polerovirus | Potato leafroll virus |
| | | Enamovirus | Pea enation mosaic |
| | | | virus-1 |
| | | Tobamovirus | Tobacco mosaic virus |
| | | Tobravirus | Tobacco rattle virus |
| | | Hordeivirus | Barley stripe mosaic |
| | | | virus |
| | | Furovirus | Soil-borne wheat |
| | | | mosaic virus |
| | | Pomovirus | Potato mop-top virus |
| | | Pecluvirus | Peanut clump virus |
| | | Benyvirus | Beet necrotic yellow |
| | | | vein virus |
| | | Idaeovirus | Raspberry bushy |
| | | | dwarf virus |
| | | Capillovirus | Apple stem grooving |
| | | | virus |
| | | Trichovirus | Apple chlorotic leaf |
| | | | spot virus |
| | | Sobemovirus | Southern bean mosaic |
| | | | virus |
| | | Umbravirus | Carrot mottle virus |
| | Tymoviridae | Tymovirus | Turnip yellow mosaic |
| | | | virus |
| | | Marafivirus | Maize rayado fino |
| | | | virus |
| | | Maculavirus | Grapevine fleck virus |
| | | Carlavirus | Carnation latent virus |
| | | Potexvirus | Potato virus X |
| | | Allexivirus | Shallot virus X |
| | | Foveavirus | Apple stem pitting |
| | | | virus |
| | | Vitivirus | Grap vine virus A |
| | | Ourmiavirus | Ourmia melon virus |

TABLE 8
Naked RNA Viruses

| | | | Type Species |
|---|---|---|---|
| Order | Family | Genus | Example |
| | Narnaviridae | Narnavirus | Saccharomyces |
| | | | cerevisiae 20SRNA |
| | | | narnavirus |
| | | Mitovirus | Cryphonectria |
| | | | parasitica mitovirus- |
| | | | 1 NB631 |

TABLE 9
Viroids

| | | | Type Species |
|---|---|---|---|
| Order | Family | Genus | Example |
| | Pospiviroidae | Pospiviroid | Potato spindle tuber |
| | | | viroid |
| | | Hostuviroid | Hop stunt viroid |
| | | Cocadviroid | Coconut cadang- |
| | | | cadang viroid |
| | | Apscaviroid | Apple scar skin |
| | | | viroid |
| | | Coleviroid | Coleus blumei |
| | | | viroid 1 |
| | Avsunviroidae | Avsunviroid | Avocado sunblotch |
| | | | viroid |
| | | Pelamoviroid | Peach latent mosaic |
| | | | viroid |

TABLE 10
Subviral Agents

| Agent | Group | Type | Subgroup/Species |
|---|---|---|---|
| Satellites | Satellite Viruses | Single-Stranded | Chronic bee- |
| | | RNA Satellite | paralysis satellite |
| | | Viruses | virus |
| | | | Tobacco necrosis |
| | | | satellite virus |
| | Satellite Nucleic | Single-Stranded | Tomato leaf curl |
| | Acids | Satellite DNAs | virus satellite DNA |
| | | Double-Stranded | satellite of |
| | | Satellite RNAs | Saccaromyces |
| | | | cerevisiae M virus |
| | | Single-Stranded | Large Satellite |
| | | Satellite RNAs | RNAs |

TABLE 11A
RNA Viruses in Disease

| Family | Virus Subfamily | Host: | Host: |
|---|---|---|---|
| (Viridae) | or Genus | Humans, Monkeys | Other Animals |
| Picorna | Entero | Enteritis, occasionally | Enteritis |
| | | CNS (polio) | |
| | Cardio | — | Encephalomyocarditis |
| | Rhino | Common Cold (many | Respiratory |
| | | serotypes) | |
| | Aphtho | — | Foot and Mouth Disease |
| Corona | — | Respiratory and enteric | Many different diseases in |
| | | | different animals |
| Toga | Alpha | Rare encephalitis | Equine, etc. encephalitis |
| | Flavi | Yellow fever, | Equine, etc. encephalitis |
| | | encephalitis | |
| | Rubi | Skin rash, German | — |
| | | Measles (rubella) | |
| | Pesti | Occasionally congenital | Mucosal disease (cattle) |
| | | diseases | |
| Retro | Type C | T-cell leukemia | Avian, murine, and other |
| | | (HTLV-1); sarcoma | animal leukemias and |
| | | (monkey) | sarcomas |
| | Type B | — | Murine mammary tumors |
| | Type D | Immune deficiency | — |
| | | (monkey) | |
| | Lenti | AIDS, encephalopathy, | Immune deficiency (cats), |
| | | Immune deficiency | maedi visna (sheep), |
| | | (monkey) | encephalopathy, arthritis |
| | | | (goats) |
| Rhabdo | Vesiculo | — | Stomatitis (cattle, swine) |
| | Lyssa | Rabies | Rabies |
| Filo | — | Hemorrhagic fever | — |
| | | (Marburg, Ebola) | |
| Arena | — | Hemorrhagic fever | Lymphocytic choriomeningitis |
| | | (Lassa) | (mice) |
| Bunya | Bunyamwera | Encephalitis (Calif. | Many diseases |
| | | Enceph.) | |
| Paramyxo | Paramyxo | Childhood respiratory, | Newcastle disease (birds) |
| | | croup (parainfluenza), | |
| | | salivary gland (Mumps) | |
| | Morbilli | Skin rash (measles) | Rinderpest (cattle), distemper |
| | | | (dogs) |
| | Pneumo | Childhood lower | |
| | | respiratory, Pneumonia | |
| | | (respiratory syncitial) | |
| Orthomyxo | Type A | Influenza (flu) | Respiratory |
| | Type B | Influenza | — |
| Reo | Orthoreo | — | — |
| | Orbi | Diarrhea | Diarrhea (blue-tongue of |
| | | | sheep) |
| | Rota | Children's diarrhea | Enteric |
| | Cytoplasmic | — | Lethal insect infection |
| | polyhedrosis | | |

TABLE 11B
DNA Viruses in Disease

| | Virus Subfamily | Host: | Host: |
|---|---|---|---|
| Family (Viridae) | or Genus | Humans, Monkeys | Other Animals |
| Parvo | — | Aplastic anemia | Enteritis (dogs, cats), |
| | | (humans), Fifth disease | Encephalopathy (rats), |
| | | (B19) | Prenatal infections |
| Hepadna | — | Hepatitis | Same (woodchucks, |
| | | | squirrels, ducks) |
| Papova | Polyoma | Malignant tumors under | Same |
| | | certain specific | |
| | | conditions; | |
| | | encephalopathy | |
| | Papilloma | Warts, carcinomas | Warts, at times |
| | | | malignant (Shope |
| | | | papilloma) |
| Adeno | (Many serotypes) | Acute respiratory | Same (occasionally |
| | | diseases, conjunctivitis | oncogenic) |
| Herpes | Alphaherpes | Skin rash: chickenpox, | — |
| | | varicella | |
| | | Cold sores, shingles | |
| | | (herpes simplex 1) | |
| | | Venereal, congential | Bovine mammalitis, etc. |
| | | (herpes simplex 2) | |
| | Betaherpes | Congenital | Respiratory and |
| | | malformations | congenital diseases |
| | | (cytomegalo) | |
| | Gammaherpes | Infectious | Marek's disease |
| | | mononucleosis, etc. | (chickens) |
| | | (Epstein-Barr) | |
| Baculo | (nuclear | — | Lethal insect infections |
| | polyhedrosis, | | |
| | granulosis) | | |
| Pox | (Many Genera) | Smallpox; Yaba | Pox, myxomatosis |
| | | (monkey) | |
| Unclassified | — | Non-A, non-B hepatitis, | — |
| | | liver cancer | |

What is claimed is:

1. A method for imparting a cell or multi-cellular organism with resistance to a viral pathogen to which the cell or multi-cellular organism is susceptible to infection, comprising the steps of:
genetically modifying the cell or multi-cellular organism to render it capable of utilizing at least one viral genomic polynucleic acid molecule or viral replication intermediate polynucleic acid molecule or viral RNA transcript molecule as a template for the template-directed polymerization of polynucleic acids by a template-directed polynucleic acid polymerase so that the viral function of the viral molecule is inhibited, wherein the cell or multi-cellular organism not so modified is at least substantially incapable of inhibiting the function of the polynucleic acid molecule, by utilizing it as a template for template-directed nucleic acid polymerization by a polynucleic acid polymerase.

2. The method of claim 1, wherein the step of genetically modifying the cell or multi-cellular organism comprises genetically modifying the cell or multi-cellular organism to express a template-directed polynucleic acid polymerase capable of utilizing the viral polynucleic acid as template.

3. The method of claim 1, wherein the step of genetically modifying the cell or multi-cellular organism further comprises, genetically modifying the cell or multi-cellular organism to produce a polynucleic acid primer molecule capable of directing a template directed polynucleic acid polymerase to utilize the viral polynucleic acid molecule as a template.

4. The method of claim 2, wherein the step of genetically modifying the cell or multi-cellular organism further comprises, genetically modifying the cell or multi-cellular organism to produce a polynucleic acid primer molecule capable of directing a template directed polynucleic acid polymerase to utilize the viral polynucleic acid as a template.

5. The method of any one of claims 1, wherein the step of genetically modifying the cell or multi-cellular organism further comprises genetically modifying the cell or multi-cellular organism to express a nuclease capable of degrading the viral polynucleic acid nucleic molecule acid which serves as a template for the polymerase, during or after template directed nucleic acid polymerization by the polymerase.

6. The method of claim 5, wherein the viral polynucleic acid is an RNA molecule and the nuclease is an RNAse H nuclease.

7. The method of claim 5, wherein the viral polynucleic acid is an RNA molecule and the nuclease is an RNAse III nuclease.

8. The method of claim 7, wherein the nuclease is E. coli RNAse III.

9. The method of claim 7, wherein the nuclease is a Dicer nuclease.

10. The method of claim 2, wherein the template-directed polynucleic acid polymerase comprises an integral nuclease or a closely associated nuclease, the nuclease being capable of degrading the viral nucleic acid which serves as a template for the polymerase, during or after template directed nucleic acid polymerization by the polymerase.

11. A method of making the expression of a RNA transcript of a preselected cellular gene responsive to viral infection of the cell wherein the viral infection provides a viral RNA-dependent polynucleic acid polymerase within the cell, comprising the steps of:
genetically modifying a cell or multi-cellular organism to express at least one primer molecule capable of directing the viral polymerase to utilize the RNA transcript of the selected gene as a template for template-directed polynucleic acid polymerization so; or
genetically modifying the cell or multi-cellular organism so that the transcript of the preselected gene comprises a functional polymerase binding site for the viral polymerase.

12. The method of claim 11, wherein the step of genetically modifying the cell or multi-cellular organism further comprises genetically modifying the cell or multi-cellular organism to express a nuclease capable of degrading the cellular RNA transcript which serves as a template for the polymerase, during or after template directed nucleic acid polymerization by the polymerase.

13. The method of claim 11 or 12, wherein the cellular RNA transcript is an mRNA molecule.

14. A method for making a cell or multi-cellular organism resistant to a viral pathogen to which the cell or multi-cellular organism is susceptible to infection, and wherein the virus provides an RNA-dependent polynucleic acid polymerase to the cell upon infection, comprising the steps of:
genetically modifying the cell or multi-cellular organism to express at least one primer molecule capable of directing the viral polymerase to utilize at one viral RNA molecule template for template-directed polynucleic acid polymerization so that the viral function of the RNA molecule is inhibited.

15. The method of claim 14, wherein the step of genetically modifying the cell or multi-cellular organism further comprises genetically modifying the cell or multi-cellular organism to express a nuclease capable of degrading the viral RNA molecule which serves as a template for the polymerase, during or after template directed nucleic acid polymerization by the polymerase.

16. The method of claim 14, wherein the viral RNA molecule is a viral mRNA transcript.

17. The method of claim 14, wherein the viral RNA transcript is a viral genomic RNA molecule or a viral replication intermediate RNA molecule.

18. A method of rendering a cell or multi-cellular organism capable of at least partially inhibiting replication of a virus infecting the cell or cells of the organism, wherein replication of the virus characteristically proceeds from a polymerase binding site (PBS) in the viral genome using a characteristic polymerase, the method comprising the steps of:
genetically modifying the cell or multi-cellular organism to express at least one molecule capable of binding to regions of the genome of the viral pathogen downstream of the characteristic primer binding site with respect to the direction of normal polymerization from the PBS to cause the template directed polymerization of nucleic acids from the downstream site.

19. The method of claim 18, wherein the polymerase is provided by the viral pathogen and wherein the at least one molecule comprises a primer molecule for primer extension by the polymerase so that extension from the characteristic primer binding site by the polymerase to form a full length characteristic viral polymerization product is prevented.

20. The method of claim 19, wherein the polymerase has an integral or closely associated nuclease which degrades the polynucleic acid strand used as a template by the polymerase as the polymerase uses the template for template directed polynucleic acid polymerization.

21. The method of claim 19, wherein the step of genetically modifying the cell or multi-cellular organism further comprises genetically modifying the cell or multi-cellular organism to express a nuclease capable of degrading the viral nucleic acid molecule which serves as a template for the polymerase, during or after template directed nucleic acid polymerization by the polymerase.

22. The method of claim 20, wherein the polymerase is a reverse transcriptase.

23. A method of inhibiting the function of a preselected polynucleic molecule in a cell, the polynucleic acid molecule characteristically serving as a template in the cell for either reverse transcription by a reverse trancriptase or RNA directed RNA polymerization by an RNA dependent RNA polymerase, comprising the steps of:
(i) if the polynucleic molecule is characteristically a template for reverse transcription, rendering the polynucleic molecule a template for RNA template directed RNA polymerization in the cell; or
(ii) if the polynucleic molecule is characteristically a template for RNA template directed RNA polymerization, rendering the polynucleic molecule a template for reverse transcription in the cell.

24. The method of claim 23, wherein the polynucleic acid molecule is a viral polynucleic acid molecule and the function inhibited is a viral function of the molecule.

25. A cell genetically modified for the regulation of cellular gene expression in response to infection of the cell by a virus, comprising
at least one genetic modification rendering at least one preselected cell-encoded transcript capable of serving as a template for the template directed polymerization of nucleic acids as a result of provision to the cell by the virus of at least one viral factor, thereby inhibiting the activity of the transcript.

26. The cell of claim 25, wherein the at least one viral factor is selected from the group consisting of a primer molecule of viral origin and a viral RNA dependent nucleic acid polymerase.

27. The method of claim 25 or 26, wherein the sequence of the gene encoding the transcript is modified, versus the corresponding unmodified form, so that the transcript comprises a primer binding site or polymerase binding site by which the transcript is functionally rendered a template for said polymerization in response to the viral infection.

28. The cell of claim 25, wherein the transcript is an mRNA transcript for an essential gene.

29. The cell of claim 25, wherein the activity of the transcript or a protein for which it codes is as a direct or indirect regulator of transcription of at least one other gene.

30. The cell of claim 29, wherein the gene codes for a transcriptional repressor protein or a transcriptional activator protein, the protein regulating the expression of at least one other cellular gene.

31. The cell of claim 30, wherein expression of the at least one other gene is increased upon inhibition of the activity of the transcript.

32. The cell of claim 31, wherein the at least one other gene codes for a toxin which is toxic to the cell.

33. The cell of claim 31, wherein the at least one other gene codes for a lethal gene which is lethal to the cell.

34. The cell of claim 31, wherein the at least one other gene codes for a protoxin to toxin converting enzyme.

35. The cell of claim 31, wherein the at least one other gene codes for a site specific DNA recombinase.

36. The cell of claim 31, wherein the at least one other gene codes for a protein product having anti-viral activity against the virus.

37. The cell of claim 31, wherein the at least one other gene is a reporter gene.

38. The cell of claim 31, wherein the at least one other gene codes for a polynucleic acid primer molecule.

39. A method for modulating the activity of a preselected polynucleic acid molecule in a cell in response to infection of the cell by at least one predetermined virus, comprising the steps of:
rendering the preselected polynucleic acid molecule a template for the template directed polymerization of nucleic acids by at least one preselected nucleic acid dependent nucleic acid polymerase, wherein said template directed polymerization modulates the activity of said molecule; and
wherein the amount of functional interaction between said molecule and said polymerase is regulated by infection of the cell by the virus.

40. The method of 39, wherein the preselected polynucleic acid is a cellular polynucleic acid molecule or a viral polynucleic acid molecule in a cell and the preselected nucleic acid dependent nucleic acid polymerase is encoded by the genome of the cell or by the genome of a virus infecting the cell

41. The method of 39, wherein the preselected polynucleic acid molecule is a viral polynucleic acid in the cell and the preselected nucleic acid dependent nucleic acid polymerase is encoded by the genome of the cell or by the genome of a virus infecting the cell.

42. The method of 40 or 41, wherein the preselected nucleic acid dependent nucleic acid polymerase is encoded by the genome of a virus infecting the cell.

43. The method of claim 40, wherein the amount of functional interaction between said molecule and said polymerase is further regulated by genetically modifying the cell to express the polymerase or to increase expression of the polymerase.

44. The method of claim 43, wherein the polymerase is not endogenous to the type of cell.

45. The method of claim 40, wherein the amount of functional interaction between said molecule and said polymerase is further regulated by genetically modifying the cell to (i) express a primer molecule capable of directing the polymerase to utilize the polynucleic acid molecule as a template or (ii) increase the expression of such a primer.

46. A method for decreasing the activity of a preselected polynucleic acid molecule present in a cell, comprising the steps of:
rendering said polynucleic acid molecule a template in the cell for the template directed polymerization of nucleic acids by at least one preselected nucleic acid dependent nucleic acid polymerase wherein said template directed polymerization diminishes the activity of said molecule in the cell,

47. A genetically-modified multi-cellular organism resistant to viral pathogenicity for at least one preselected virus versus the multi-cellular organism not so modified, comprising:
(i) cells wherein the modification causes the activity of at least one preselected viral polynucleic acid molecule to be diminished in the cells as a result of the preselected viral polynucleic acid being rendered a template for the template directed polymerization of nucleic acid molecules within the cell; or
(ii) cells wherein the modification causes the activity of at least one preselected cellular RNA transcript to be diminished in response to infection by the virus as a result of the preselected cellular transcript being rendered a template for the template directed polymerization of nucleic acid molecules within the cell, resulting in resistance of the cell and/or the multi-cellular organism as a whole to the pathogenicity of the virus.

48. The genetically-modified multi-cellular organism of claim 47, wherein the organism is a plant.

49. The seed of the plant of claim 48

50. The plant of claim 48, cultivated in an environment wherein exposure to the virus may occur.

51. The genetically-modified multi-cellular organism of claim 47, wherein the organism is a non-human animal.

52. The genetically-modified multi-cellular organism of claim 47, wherein the organism is a mammal.

53. The genetically-modified multi-cellular organism of claim 47, wherein the organism is a crustacean.

54. The genetically-modified multi-cellular organism of claim 47, wherein the organism is a fish.

55. The genetically-modified multi-cellular organism of claim 47, wherein the organism is avian.

56. A stem cell capable of generating into the genetically modified multi-cellular organism of claim 51.

57. A genetically modified plant resistant to infection by at least one preselected virus, wherein
the plant is genetically modified to render at least one preselected polynucleic acid molecule of the virus a template for the template directed polymerization of nucleic acids by at least one preselected nucleic acid dependent nucleic acid polymerase wherein said template directed polymerization diminishes the viral function of said viral polynucleic acid molecule; and
wherein the amount of functional interaction between said polynucleic acid molecule of the virus and said polymerase, is regulated by infection of the cells of the plant by the virus.

58. The seed of a plant of claim 57.

59. A cell capable of regeneration into the plant of claim 57.

60. The plant of claim 57, cultivated in an environment wherein exposure to the virus may occur.
